(19) United States
(12) Patent Application Publication
(10) Pub. No.: US 2013/0272978 A1
(43) Pub. Date: Oct. 17, 2013

(54) DIHYDROXYACETONE MONOETHERS

(71) Applicants: Philipp Buehle, Zwingenberg (DE); Thomas Rudolph, Darmstadt (DE)

(72) Inventors: Philipp Buehle, Zwingenberg (DE); Thomas Rudolph, Darmstadt (DE)

(73) Assignee: MERCK PATENT GMBH, Darmstadt (DE)

(21) Appl. No.: 13/995,180

(22) PCT Filed: Nov. 30, 2011
(86) PCT No.: PCT/EP11/06006

§ 371 Date: Jun. 18, 2013

(30) Foreign Application Priority Data
Dec. 22, 2010 (DE) 10 2010 055 656.4

(51) Int. Cl. C07C 49/175; C07C 45/60; A61Q 17/04; A61K 8/35

(52) U.S. Cl. CPC C07C 49/175; A61K 8/35; C07C 45/60; A61Q 17/04
USPC 424/59; 568/308; 568/414; 568/404

(57) ABSTRACT

The present invention relates to the use of dihydroxyacetone monoethers as self-tanning substance, to preparations comprising dihydroxyacetone monoethers, and to certain dihydroxyacetone monoethers and to a process for the preparation thereof.

The present invention relates to the use of dihydroxyacetone monoethers as self-tanning substance, to preparations comprising dihydroxyacetone monoethers, and to certain dihydroxyacetone monoethers and to a process for the preparation thereof.

The trend away from refined paleness towards “healthy, sporty brown skin” has been uninterrupted for years. In order to achieve a tanned complexion, people expose their skin to sunlight, since this causes pigmentation due to melanin formation. However, the UV radiation in sunlight also has a damaging effect on the skin. Besides acute damage (sunburn), long-term damage occurs on excessive irradiation with light from the UVB region (wavelength 280-320 nm), such as, for example, an increased risk of contracting skin cancer. Excessive exposure to UVB and UVA radiation (wavelength: 320-400 nm) generates highly reactive free-radical species, which multiply further even after termination of the irradiation, and wrinkling and skin ageing occur as a consequence thereof.

Tanning (pigmentation) of the skin offers natural protection against the adverse consequences of sunlight. The epidermis contains individual pigment-forming cells, the melanocytes, besides the basal cells in its lowest layer, the basal layer. UV light stimulates the production of melanin in these cells, which is transported into the kerantinocytes (horny cells), where it becomes visible as a brown skin colour. Melanin protects the cell nuclei against further irradiation and the adverse effects it causes on the cell DNA.

Depending on the chemical composition of the pigments formed biochemically, a distinction is made between brownish-black eumelanin and reddish-yellow pheomelanin. The skin hue observed is determined by the ratio of these two types of melanin.

This pigment formation starting from the amino acid tyrosine is initiated predominantly by UVB radiation and is known as “indirect pigmentation”. Its development runs over a number of days; the suntan obtained in this way lasts a few weeks. In the case of “direct pigmentation”, which commences with the solar irradiation, predominantly colourless melanin precursors are oxidised by UVA radiation to dark-coloured melanin. Since this oxidation is reversible, it results in skin tanning which only lasts briefly.

Artificial tanning of the skin can be produced externally with the aid of make-up and orally by taking carotenoids.

Much more popular, however, is artificial tanning of the skin which can be achieved by the application of so-called self-tanners.

The substance most frequently employed worldwide for these purposes is 1,3-dihydroxyacetone (DHA), which is used in an amount of 700t/a. Self-tanners can be reacted with the proteins and amino acids of the horny layer of the skin in the sense of a Maillard reaction or via a Michael addition, where polymers which give the skin a brownish hue form via a reaction route which has not yet been clarified completely. This reaction is complete after about 24 hours. The tan achieved in this way cannot be washed off and is only removed with the normal skin desquamation.

Various substances are already known which are employed as self-tanners and which are described below.

DHA is a water-soluble crystalline solid which is unstable under neutral to basic conditions. This instability is also accompanied by the development of cosmetically undesired malodours.

A problem which can frequently occur on use with self-tanner substances, in particular with 1,3-dihydroxyacetone, is that the natural tanning of the skin is discoloured towards orange by the dominance of the yellow component. This is frequently regarded by many users as a disadvantage and perceived as unnatural.

There therefore continues to be a demand for dermatologically tolerated skin-colouring substances which are suitable for use in cosmetic and/or dermatological preparations or medical products.

The object on which the present invention is based therefore consisted in the provision of self-tanning substances having improved properties, in particular with respect to a more natural skin shade.

Surprisingly, it has now been found that certain dihydroxyacetone monoethers are self-tanning substances which are capable of developing a more natural shade on the skin and accelerating the tanning reaction.

International application WO 2006/018104 describes compounds of the general formula

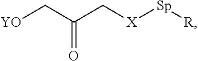

where X stands for O, S(O)ₘ or NR¹; Y stands for H, —SiR²R³R⁴ or —[Si(R²)₂]_{q}SiR³R⁴R⁵ or —Sp-R; R¹ stands for H, C₁₋₂₄-alkyl or R; R², R³, R⁴ and R⁵ stand for 1-30-alkyl; Sp stands for —(CH₂)ₙ—, —(CH₂)ₙ—C(═O)—(CH₂)ₒ— or —(CH₂)ₙ—C(═O)—(CH₂)ₒ—X—(CH₂)ₚ—; m stands for 0, 1 or 2; n, o, p stand for 0 to 24; R stands for a substituent which absorbs UV radiation. This document furthermore discloses the cosmetic use of these compounds as light-protection filters. However, the fact that certain compounds falling under this formula can also act as self-tanning substance is not mentioned or suggested in this application.

WO 2006/024361 A1 describes the use of dimeric dihydroxyacetone monoester derivatives as precursors of self-tanning compounds. EP 0709081 A1 and EP 0796838 A1 disclose preparations comprising dihydroxyacetone ester derivatives which are precursors of dihydroxyacetone. None of these documents discloses the use of dihydroxyacetone monoether derivatives as self-tanning substance.

The present invention therefore relates firstly to the use of a compound of the formula (I)

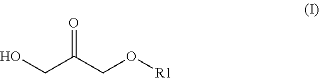

as self-tanning substance,
where R1 stands for a branched or unbranched alkyl radical having 1 to 20 C atoms, a branched or unbranched alkenyl radical having 2 to 20 C atoms or a branched or unbranched alkynyl radical having 2 to 20 C atoms, where one or more CH₂ groups which are not adjacent and are not directly adjacent to the O atom of the formula (I) may be replaced by C═O or —O—, where the radicals may be substituted by one or more OH groups, by one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms.

The compounds of the formula (I) may also be present here in the form of their dimeric derivatives of the formula (II)

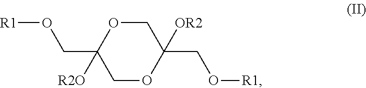

where R2 stands for H or a branched or unbranched alkyl radical having 1 to 20 C atoms and R1 is as defined for formula (I). R2 preferably stands for methyl or ethyl. The compounds of the formula (II) can be converted into the corresponding monomeric DHA ethers of the formula (I), for example by hydrolysis in the formulation, also after addition of an activating reagent (for example an acid), or by hydrolysis on the skin owing to the natural acidic pH of the skin.

R1 preferably stands for a branched or unbranched alkyl radical having 1 to 20 C atoms,

where one or more CH₂ groups which are not adjacent and are not directly adjacent to the O atom of the formula (I) may be replaced by C═O or —O—, where the radicals may be substituted by one or more OH groups, by one or more cyclic alkyl radicals having 3 to 8 C atoms and/or by one or more aromatic ring systems having 5 to 6 C atoms, where the one or more aromatic ring systems may be substituted by one or more groups selected from —OH and —OR, and where R is defined as described above.

R1 particularly preferably stands for a branched or unbranched alkyl radical having 1 to 20 C atoms,

where one or more CH₂ groups which are not adjacent and are not directly adjacent to the O atom of the formula (I) may be replaced by C═O or —O—,
where the radicals may be substituted by one or more OH groups, by one or more cyclic alkyl radicals having 5 to 6 C atoms and/or by one or more aromatic ring systems having 5 to 6 C atoms, where the one or more aromatic ring systems may be substituted by one or more groups —OR,
and where R is defined as described above.

In the above-mentioned embodiments, the branched or unbranched alkyl radical preferably has 1 to 17 C atoms.

The cyclic alkyl radical having 5 to 6 C atoms is preferably a cyclohexyl radical. The aromatic ring system having 5 to 6 C atoms is preferably a phenyl radical. R preferably stands for a branched or unbranched alkyl radical having 1 to 8 C atoms, particularly preferably for methyl or ethyl.

R1 is very particularly preferably selected from the following radicals: benzyl, 2-ethylhexyl, unbranched or branched hexyl, unbranched or branched heptyl, unbranched or branched pentadecyl, unbranched or branched heptadecyl, cyclohexylmethyl, cyclohexylethyl, phenylethyl, methoxyphenylmethyl, 2,3-dihydroxypropyl, 2-oxy-3-hydroxypropyl, hydroxyethyl, hydroxymethyl, 2-hydroxypropyl and 2-hydroxyethoxyethyl.

In accordance with the invention, the compounds of the formula (I) are preferably selected from the compounds of the formula (Ia) to (Ik)

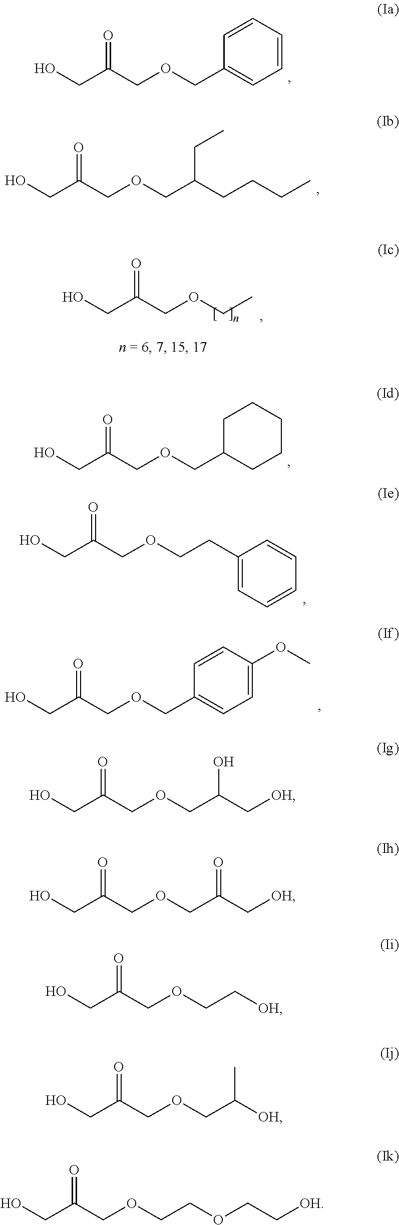

Particular preference is given here to the compounds (Ia) to (If), very particular preference is given to the compounds (Ia) to (Id).

In a further possible embodiment, R1 preferably stands for a branched or unbranched alkyl radical having 1 to 20 C atoms,

where the radicals may be substituted by one or more OH groups, by one or more cyclic alkyl radicals having 5 to 6 C atoms and/or by one or more aromatic ring systems having 5 to 6 C atoms, where the one or more aromatic ring systems may be substituted by one or more groups selected from —OH and —OR,
and where R is defined as described above.

In this embodiment, R1 particularly preferably stands for a branched or unbranched alkyl radical having 1 to 20 C atoms,

where the radicals may be substituted by one or more cyclic alkyl radicals having 5 to 6 C atoms and/or by one or more aromatic ring systems having 5 to 6 C atoms,
where the one or more aromatic ring systems may be substituted by one or more groups selected from —OH and —OR,
and where R is defined as described above.

Preferred embodiments of the cyclic alkyl radicals, of the aromatic ring system, and of the radical R are defined here as described above.

Very particularly preferred compounds of the formula (I) in this case are the compounds of the formula (Ia), (Ib), (Ic), (Id), (Ie) and (If).

For the purposes of the present invention, a branched or unbranched (straight-chain) alkyl radical having 1 to 8 C atoms is, for example, methyl, ethyl, isopropyl, propyl, butyl, sec-butyl or tert-butyl, pentyl, isopentyl, 1-, 2- or 3-methylbutyl, 1,1-, 1,2- or 2,2-dimethylpropyl, 1-ethylpropyl, 1-ethyl-1-methylpropyl, 1-ethyl-2-methylpropyl, 1,1,2- or 1,2,2-trimethylpropyl, 1,1-, 1,2-, 1,3-, 2,2-, 2,3- or 3,3-dimethylbutyl, 1- or 2-ethylbutyl, 1-, 2-, 3- or 4-methylpentyl, hexyl, heptyl, 1-ethylpentyl, octyl or 1-ethylhexyl.

Besides the C1 to C8-alkyl radicals listed above, an alkyl radical having 1 to 17 C atoms can, for example, also be nonyl, decyl, undecyl, dodecyl, tridecyl, tetradecyl, pentadecyl, hexadecyl or heptadecyl.

Besides the radicals listed above, an alkyl radical having 1 to 20 C atoms is also taken to mean octadecyl, nonadecyl or eicosyl.

In accordance with the invention, an alkenyl radical can contain one or more double bonds. A branched or unbranched alkenyl group having 2 to 20 C atoms is, for example, allyl, vinyl, propenyl, 2- or 3-butenyl, isobutenyl, sec-butenyl, 2-methyl-1- or 2-butenyl, 3-methyl-1-butenyl, 1,3-butadienyl, 2-methyl-1,3-butadienyl, 2,3-dimethyl-1,3-butadienyl, 1-, 2-, 3- or 4-pentenyl, isopentenyl, hexenyl, heptenyl or octenyl, —C₉H₁₇, —C₁₀H₁₉ to —C₂₀H₃₉.

An alkynyl radical can contain one or more triple bonds. Examples of a branched or unbranched alkynyl group having 2 to 20 C atoms are ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇.

For the purposes of the invention, a cyclic alkyl having 3 to 8 C atoms denotes saturated and partially unsaturated non-aromatic cyclic hydrocarbon groups which contain 3 to 8 C atoms and may be unsubstituted or substituted. The bonding to the alkyl, alkenyl or alkynyl radical of the formula (I) can take place via any ring member of the cycloalkyl group. Examples of suitable cycloalkyl radicals are cyclopropyl, cyclobutyl, cyclopentyl, cyclohexyl, cycloheptyl, cyclooctyl, cyclopentenyl, cyclopentadienyl, cyclohexenyl and cyclooctadienyl. A cyclic alkyl radical having 6 C atoms is preferably cyclohexyl.

An aromatic ring system having 5 to 6 C atoms in the sense of the present invention may be in substituted or unsubstituted form. The bonding to the alkyl, alkenyl or alkynyl radical of the formula (I) can take place via any ring member of the aromatic ring system. Examples of such aromatic ring systems having 5 to 6 C atoms are phenyl, methoxyphenyl, 2,6-dimethoxyphenyl, 3,5-dimethoxyphenyl, 2,4-dimethoxyphenyl, 2,4,6-trimethoxyphenyl or 2,3,4-trimethoxyphenyl.

Preference is given to aromatic ring systems which may be substituted by one or more —OR groups. Particular preference is given to phenyl and methoxyphenyl.

The compounds of the formula (Ia), (Ie) and (If) may have the advantage that, through the cleaving-off of the ether radical, they can, as corresponding alcohol, liberate odoriferous substances during the tanning reaction. Phenethyl alcohol (obtainable by cleaving the ether radical off the compound of the formula (Ie)) is perceived, for example, as a rose/hyacinth-like fragrance. These liberated odoriferous substances are advantageous since the odour perceived during the tanning reaction is usually perceived as negative.

The compounds of the formula (I) can be prepared starting from dimeric dihydroxyacetone: the synthesis of 2,5-diethoxy-2,5-bis(hydroxymethyl)-1,4-dioxane is carried out as disclosed in Jung et al., Journal of Organic Chemistry, 1994, 7182. The free OH groups of the ethoxylated dimer can subsequently be modified by a conventional etherification reaction using an alkyl halide or an alkyl tosylate or mesylate.

In a final step, the monomeric compound of the formula (I) can then be obtained by acidic hydrolysis, for example using 95% sulfuric acid.

The synthesis is illustrated in the following reaction scheme:

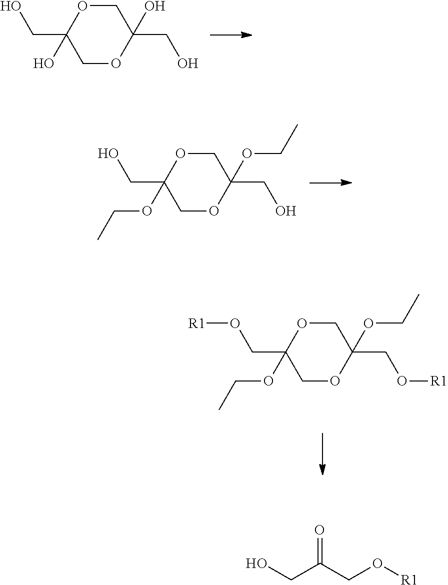

The person skilled in the art is presented with absolutely no difficulties here in selecting the suitable reaction conditions for this reaction.

Dimeric dihydroxyacetone and the other reactants in the synthesis are commercially available or accessible by syntheses which are known to the person skilled in the art from the literature.

Alternatively, the preparation can also be carried out biotechnologically starting from the corresponding glycerin ether derivatives.

The compounds of the formula (I) can be employed in self-tanning products, for example as an alternative to DHA and/or erythrulose.

An advantage on use of the compounds of the formula (I) over DHA as self-tanning substance is, for example, that they facilitate the achievement of a more natural or different skin shade. Compounds of the formula (Ic) promote, for example, a darker coloration of the skin. With the aid of compounds of the formula (Ia), a more natural skin tone can be produced by the shift towards a redder shade. A further advantage is the tanning reaction which is accelerated by the compounds of the formula (I).

Furthermore, the compounds of the formula (I) can also be used in combination with DHA and/or erythrulose.

The present invention therefore furthermore also relates to the use of a compound of the formula (I) as self-tanning substance, characterised in that the use takes place together with DHA and/or erythrulose.

The combination preferably takes place with DHA.

A combination of this type can have, for example, a positive influence on the shade achieved in the tanning reaction. The resultant skin tone can thus be matched accurately to the specific wishes of the user through the specific choice of one or more certain compounds of the formula (I) and by setting a certain mixing ratio of the compounds of the formula (I) to DHA and/or erythrulose. A further advantage of the combination may be that the compounds of the formula (I) can accelerate the tanning reaction by dihydroxyacetone or by a mixture of self-tanning substances comprising dihydroxyacetone. This enables a shortening of the application time.

Throughout the document, the term self-tanner or self-tanning substance or self-tanner substance is used synonymously. These terms denote a substance which colours the skin and can react with the amino acids of the protein-containing matrix in the sense of a Maillard reaction or via a Michael addition and thus form melanoids. The melanoids are yellow-brown to virtually black organic compounds which can essentially arise through the reaction of carbonyl groups with amino or thio functions. The principle of colouring with formation of melanoids is the basic colouring principle of the self-tanning substances.

The use according to the invention preferably takes place non-therapeutically.

The present invention furthermore relates to a preparation comprising at least one compound of the formula (I)

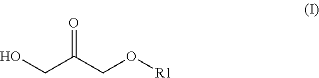

where R1 stands for a branched or unbranched alkyl radical having 1 to 20 C atoms, a branched or unbranched alkenyl radical having 2 to 20 C atoms or a branched or unbranched alkynyl radical having 2 to 20 C atoms, where one or more CH₂ groups which are not adjacent and are not directly adjacent to the O atom of the formula (I) may be replaced by C═O or —O—, where these radicals may be substituted by one or more OH groups, one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms.

The compounds of the formula (I) may also be present in the preparation in the form of their dimeric derivatives of the formula (II)

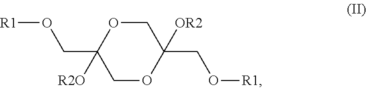

where R2 stands for H or a branched or unbranched alkyl radical having 1 to 20 C atoms and R1 is as defined for formula (I). R2 preferably stands for methyl or ethyl.

The preparations may furthermore comprise a certain proportion of the correspondingly substituted DHA diethers.

The preparations here are usually preparations which can be applied topically, for example cosmetic or dermatological formulations or medical products. In this case, the preparations comprise a cosmetically or dermatologically suitable vehicle and, depending on the desired property profile, optionally further suitable ingredients. In the case of pharmaceutical preparations, the preparations in this case comprise a pharmaceutically tolerated vehicle and optionally further pharmaceutical active compounds.

In the sense of the present invention, the term composition or formulation is also used synonymously alongside the term preparation.

“Can be applied topically” in the sense of the invention means that the preparation is applied externally and locally, i.e. that the preparation must be suitable for, for example, application to the skin.

The preparations may include or comprise, essentially consist of or consist of the said requisite or optional constituents. All compounds or components which can be used in the preparations are either known and commercially available or can be synthesised by known processes.

The preparation is preferably a cosmetic or pharmaceutical preparation; the preparation is particularly preferably a cosmetic preparation.

The at least one compound of the formula (I) is employed in the preparations according to the invention in amounts of 0.01 to 20% by weight, preferably in amounts of 0.05 to 10% by weight, particularly preferably in amounts of 0.1% by weight to 5% by weight and very particularly preferably in amounts of 0.5 to 2% by weight, based on the total amount of the preparation. The person skilled in the art is presented with absolutely no difficulties here in selecting the amounts appropriately depending on the intended action of the preparation.

Furthermore, the preparations according to the invention may comprise at least one further self-tanning substance as further ingredient. Advantageous self-tanning substances which can be employed are, inter alia:

1,3-dihydroxyacetone, glycerolaldehyde, hydroxymethylglyoxal, γ-dialdehyde, erythrulose, 6-aldo-D-fructose, ninhydrin, 5-hydroxy-1,4-naphtoquinone (juglone) or 2-hydroxy-1,4-naphtoquinone (lawsone). Very particular preference is given to 1,3-dihydroxyacetone, erythrulose or a combination thereof.

The further self-tanning substance is preferably selected from DHA and erythrulose.

The at least one further self-tanning substance is preferably present in the preparation in an amount of 0.01 to 20% by weight, particularly preferably in an amount of 0.5 to 15% by weight and very particularly preferably in an amount of 1 to 5% by weight, based on the total amount of the preparation.

Preparations having self-tanner properties, in particular those which comprise dihydroxyacetone, tend towards malodours on application to the human skin, which are thought to be caused by degradation products of dihydroxyacetone itself or by products of side reactions and which are regarded as unpleasant by some users. It has been found that these malodours are prevented on use of formaldehyde scavengers and/or flavonoids. The preparation according to the invention may therefore preferably also comprise formaldehyde scavengers and optionally flavonoids for improving the odour. However, the compounds of the formula (I) claimed for preparations according to the invention may themselves also contribute to the improvement in odour.

The formaldehyde scavenger is preferably selected from the group alkali-metal, alkaline-earth metal or ammonium disulfite.

The preparations according to the invention may particularly preferably comprise flavonoids for improving the odour and for accelerating tanning.

The flavonoid here additionally acts as stabiliser for the self-tanner or the self-tanning substances and/or reduces or prevents or improves storage-dependent malodours, which may also arise due to additives or assistants present.

It is preferably a flavonoid in which one or more phenolic hydroxyl groups have been blocked by etherification or esterification. For example, hydroxyethyl-substituted flavonoids, such as, preferably, troxerutin, troxequercetin, troxeisoquercetin or troxeluteolin, and flavonoid sulfates or flavonoid phosphates, such as, preferably, rutin sulfates, have proven to be particularly highly suitable flavonoids here. Particular preference is given in the sense of the use according to the invention to rutin sulfate and troxerutin. Very particular preference is given to the use of troxerutin.

The preferred flavonoids have a non-positively charged flavan skeleton. It is thought that metal ions, such as, for example, Fe²⁺/Cu²⁺, are complexed by these flavonoids and autooxidation processes in the case of fragrances or compounds whose degradation results in malodours are thus prevented or reduced.

Besides the compounds of the formula (I), preferred preparations additionally comprise at least one UV filter.

Organic UV filters, so-called hydrophilic or lipophilic sun-protection filters, which are effective in the UVA region and/or UVB region and(/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidene-dicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzo-triazolyl)tetramethylbutylphenol in solid form, for example marketed by Fairmount Chemical under the name “MIXXIM BB/100”, or in micronised form as an aqueous dispersion, for example marketed by BASF under the name “Tino-sorb M”.

Triazine derivatives: ethylhexyltriazone, for example marketed under the name “Uvinul T150” by BASF, diethylhexylbutamidotriazone, for example marketed under the name “Uvasorb HEB” by Sigma 3V, 2,4,6-tris(diisobutyl 4′-aminobenzalmalonate)-s-triazine or 2,4,6-tris(biphenyl)-1,3,5-triazine. marketed as Tinosorb A2B by BASF, 2,2′-[6-(4-methoxyphenyl)-1,3,5-triazine-2,4-diyl]bis[5-(2-ethylhexyl)oxy]phenol, marketed as Tinosorb S by BASF, N2,N4-bis[4-[5-(1,1-dimethylpropyl)-2-benzoxazolyl]phenyl]-N-6-(2-ethylhexyl)-1,3,5-triazine-2,4,6-triamine marketed as Uvasorb K 2A by Sigma 3V.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

Piperazine derivatives, such as, for example, the compound

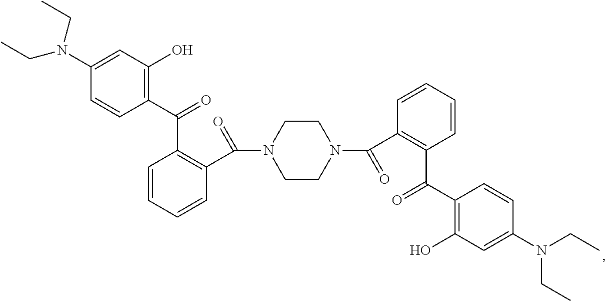 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

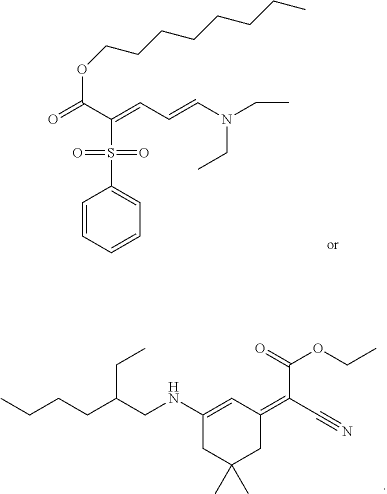

It is also possible to use UV filters based on polysiloxane copolymers having a random distribution in accordance with the following formula, where, for example, a=1.2; b=58 and c=2.8:

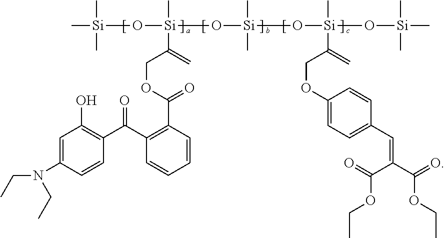

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidene-dicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-bis[5-1 (dimethylpropyl)benzoxazol-2-yl(4-phenyl)imino]-6-(2-ethylhexyl)imino-1,3,5-triazine and mixtures thereof.

These organic UV filters are generally incorporated into formulations in an amount of 0.01 percent by weight to 20 percent by weight, preferably 1% by weight—10% by weight.

Besides the compounds of the formula (I) and the optional organic UV filters, as described above, the preparations may comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex® T-2000, Eusolex®T-AQUA, Eusolex®T-AVO, Eusolex®T-OLEO), zinc oxides (for example Sachtotec®), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FF-Pharma.

It may furthermore be preferred for the preparations to comprise inorganic UV filters which have been aftertreated by conventional methods, as described, for example, in Cosmetics & Toiletries, February 1990, Vol. 105, pp. 53 64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanol-amines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
aftertreated micronised titanium dioxides with aluminium oxide and/or aluminium stearate/laurate aftertreatment, such as, for example, Microtitanium Dioxide MT 100 T from Tayca, Eusolex T-2000 from Merck,
aftertreated micronised titanium dioxides with iron oxide and/or iron stearate aftertreatment, such as, for example, the product “Microtitanium Dioxide MT 100 F” from Tayca,
aftertreated micronised titanium dioxides with silicon dioxide, aluminium oxide and silicone aftertreatment, such as, for example, the product “Microtitanium Dioxide MT 100 SAS”, from Tayca,
aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Micro-titanium Dioxide USP Grade Hydrophobic” from Color Techniques.

The combination with the following products may furthermore also be advantageous:
untreated zinc oxides, such as, for example, the product Z-Cote from BASF (Sunsmart), Nanox from Elementis
aftertreated zinc oxides, such as, for example, the following products:
   “Zinc Oxide CS-5” from Toshibi (ZnO aftertreated with polymethyl-hydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilesquioxane);
   Untreated cerium oxide micropigment, for example with the name “Colloidal Cerium Oxide” from Rhone Poulenc
   Untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide. zinc oxide mixtures, such as, for example, the product UV-Titan M261 from Sachtleben.

These inorganic UV filters are generally incorporated into the preparations in an amount of 0.1 percent by weight to 25 percent by weight, preferably 2% by weight—10% by weight.

By combination of one or more of the said compounds having a UV filter action, the protective action against harmful effects of the UV radiation can be optimised.

All said UV filters can also be employed in encapsulated form. In particular, it is advantageous to employ organic UV filters in encapsulated form.

The capsules in preparations to be employed in accordance with the invention are preferably present in amounts which ensure that the encapsulated UV filters are present in the preparation in the percent by weight ratios indicated above.

The preparations described, which in accordance with the invention comprise the at least one compound of the formula (I), may furthermore also comprise coloured pigments, where the layer structure of the pigments is not limited.

The coloured pigment should preferably be skin-coloured or brownish on use of 0.5 to 5% by weight. The selection of a corresponding pigment is familiar to the person skilled in the art.

Preferred preparations may likewise comprise at least one further cosmetic active compound, for example selected from antioxidants, anti-ageing, anti-wrinkle, anti-dandruff, anti-acne, anti-cellulite active compounds, deodorants, skin-lightening active compounds or vitamins.

The protective action of preparations against oxidative stress or against the effect of free radicals can be improved if the preparations comprise one or more antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles, (for ecample urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thio-dipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA, pentasodium ethylenediamine tetramethylene phosphonate and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example seleno-methionine), stilbenes and derivatives thereof (for example stilbene oxide, trans-stilbene oxide).

Suitable antioxidants are also compounds of the formulae A or B

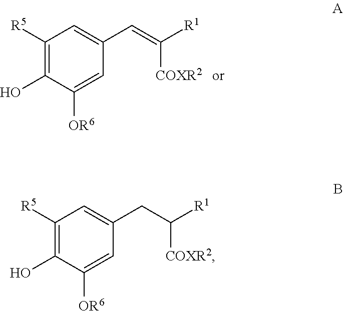

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H, linear or branched alkyl having 1 to 8 C atoms or linear or branched alkoxy having 1 to 8 C atoms and
R⁶ denotes linear or branched alkyl having 1 to 8 C atoms,
   preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-di-methoxybenzyl)malonate (for example RonaCare® AP).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such preparations with the compounds according to the invention in percent by weight ratios in the range from 1000:1 to 1:1000, preferably in percent by weight ratios of 100:1 to 1:100.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology&Medicine 2001, 31(7), 869-881, have investigated the pH dependence of the antioxidant action of hydroxyflavones. Quercetin exhibits the highest activity amongst the structures investigated over the entire pH range.

Suitable anti-ageing active compounds, in particular for skin-care preparations, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myoinositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N.-acetylornithine, trimethylamine N-oxide di-myoinositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof.

Additionally, anti-aging active compounds which can be used are products from Merck, such as, for example, 5,7-dihydroxy-2-methylchromone, marketed under the trade name RonaCare®Luremine, RonaCare® Isoquercetin, RonaCare®Tilirosid or RonaCare® Cyclopeptide 5.

The preparations may also comprise one or more skin-lightening active compounds or synonymously depigmentation active compounds or melanogenesis inhibitors. Skin-lightening active compounds can in principle be all active compounds known to the person skilled in the art. Examples of compounds having skin-lightening activity are hydroquinone, kojic acid, arbutin, aloesin, niacinamide, azelaic acid, elagic acid, mulberry extract, magnesium ascorbyl phosphate, liquorice extract, emblica, ascorbic acid or rucinol.

Preparations comprising a combination of the compounds of the formula (I) with skin-lightening active compounds can have, for example, a contrast-reducing effect. In accordance with the invention, the term contrast reduction means that an inhomogeneous skin coloration is reduced and the contrast between more strongly and less strongly coloured skin areas is thus reduced. An inhomogeneous skin coloration of this type may occur due to inhomogeneous pigmentation and/or a different distribution of the horny skin.

The combination of tanning substances whose mode of action is based on the Maillard reaction or Michael addition with melanogenesis-inhibiting substances has the effect that skin areas which are already hyperpigmented lose their high melanin concentration and the shade produced by the colorant on the skin surface becomes established over a large area.

The preparations to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the flavonoid-containing premixes or preparations in ranges from 0.01 to 5.0% by weight, based on the total weight. Nutrition-physiological applications are oriented towards the respective recommended vitamin requirement.

The retinoids described are at the same time also effective anti-cellulite active compounds. A likewise known anti-cellulite active compound is caffeine.

The present invention also relates to a process for the preparation of a preparation, as described above, characterised in that at least one compound of the formula (I) is mixed with a vehicle which is suitable for topical preparations and optionally with assistants and or fillers. Suitable vehicles and assistants or fillers are described in detail in the following part.

The said constituents of the preparation can be incorporated in the usual manner, with the aid of techniques which are well known to the person skilled in the art.

The cosmetic and dermatological preparations can be in various forms. Thus, they can be, for example, a solution, a water-free preparation, an emulsion or microemulsion of the water-in-oil (W/O) type or of the oil-in-water (O/W) type, a multiple emulsion, for example of the water-in-oil-in-water (W/O/W) or O/W/O type, a gel, a solid stick, an ointment or also an aerosol. Preference is given to emulsions. O/W emulsions are particularly preferred. Emulsions, W/O emulsions and O/W emulsions can be obtained in the usual manner.

The following, for example, may be mentioned as application form of the preparations to be employed: solutions, suspensions, emulsions, PIT emulsions, pastes, ointments, gels, creams, lotions, powders, soaps, surfactant-containing cleansing preparations, oils, aerosols plasters, compresses, bandages and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, pro-pane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexanecarbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxy-ethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkyl-amidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of sub-stances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkane-carboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, iso-propyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

In a preferred embodiment, the preparations to be employed comprise hydrophilic surfactants. The hydrophilic surfactants are preferably selected from the group of the alkylglucosides, acyl lactylates, betaines and coconut amphoacetates.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laurethyl-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan mono-oleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty bodies, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, use is generally made of the customary propellants, such as alkanes, fluoroalkanes and chlorofluoroalkanes, preferably alkanes.

The present invention furthermore relates to compounds of the formula (I)

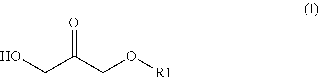

where R1 stands for a branched or unbranched alkyl radical having 1 to 20 C atoms, a branched or unbranched alkenyl radical having 2 to 20 C atoms or a branched or unbranched alkynyl radical having 2 to 20 C atoms,
where one or more CH₂ groups which are not adjacent and are not directly adjacent to the O atom of the formula (I) may be replaced by C═O or —O—, where these radicals may be substituted by one or more OH groups, one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms;
and where the following compounds are excluded:

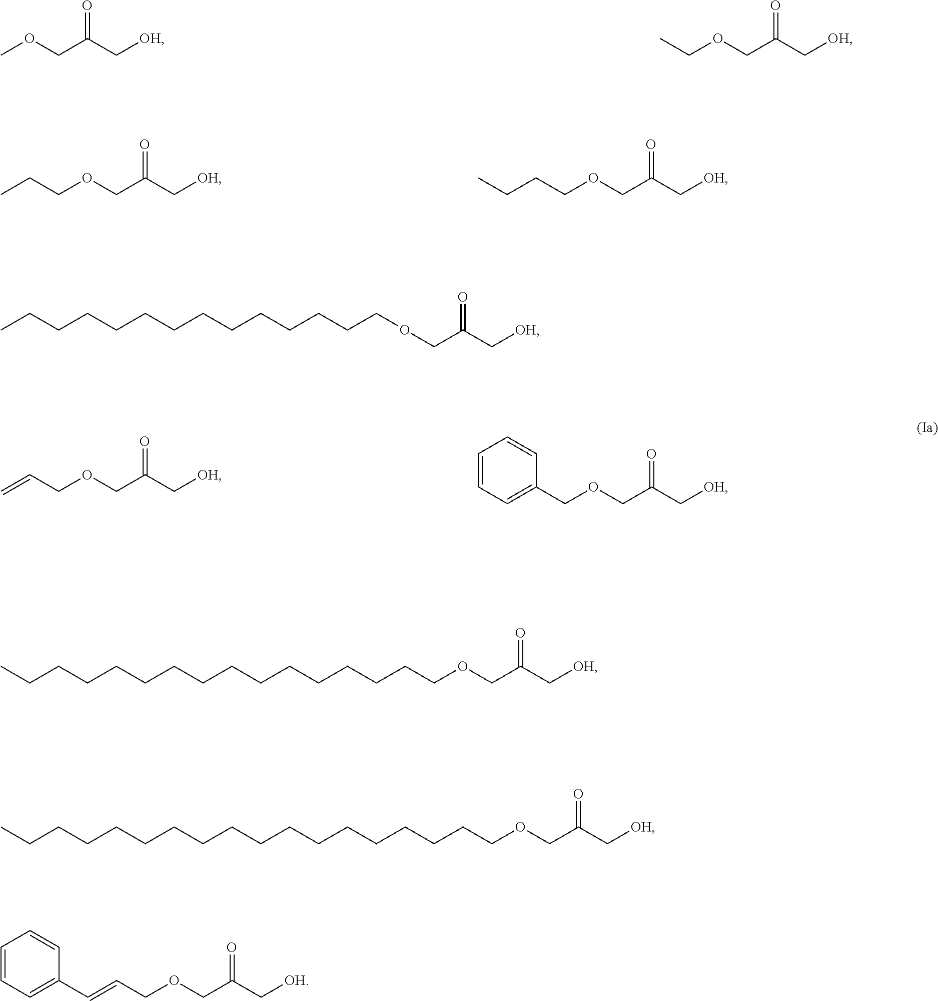

The preferred embodiments of the radicals R1 and R of the compounds according to the invention are defined here as described above.

Particular preference is given to compounds of the formula (I), characterised in that the compounds are selected from the compounds of the formulae (Ib) to (Ik)

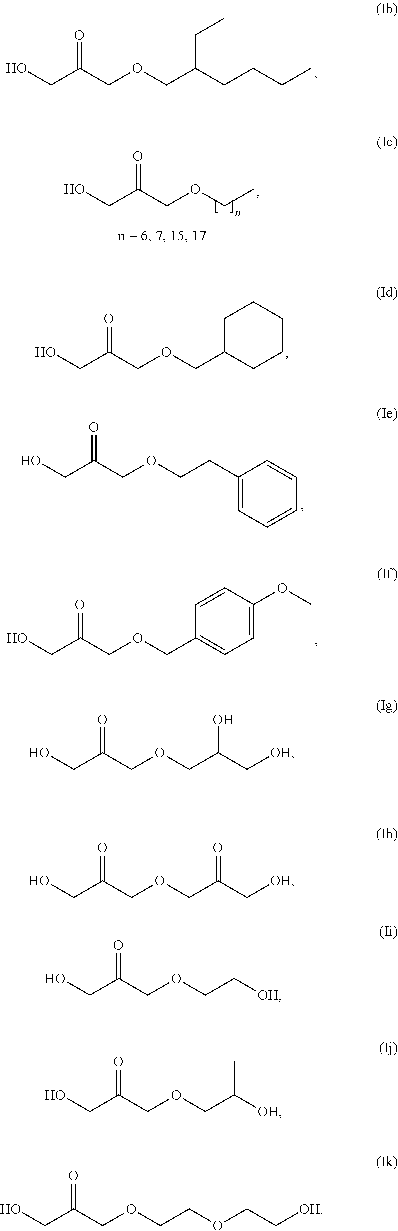

The present invention furthermore also relates to a process for the preparation of a compound of the formula (I) as defined above, characterised in that 2,5-diethoxy-2,5-bis(hydroxymethyl)-1,4-dioxane is reacted with a compound of the formula R1-X and subsequently subjected to acidic hydrolysis, where R1 is defined as described above and X stands for Cl, Br, I, OSO₂CH₃ (O-mesyl) or OSO₂C₆H₄CH₃ (Otosyl).

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference.

Further important features and advantages of the invention arise from the sub-claims and from the examples.

Preferred embodiments of the invention are described in the examples and are explained in greater detail in the following description without restricting the scope of the present invention.

### EXAMPLES

### Example 1a

### Synthesis of Compound (Ia)

### Synthesis of 2,5-diethoxy-2,5-bis(hydroxymethyl)-1,4-dioxane

2,5-Diethoxy-2,5-bis(hydroxymethyl)-1,4-dioxane is synthesised as disclosed in Jung et al., Journal of Organic Chemistry, 1994, 7182:

500 ml of anhydrous ethanol 3 ml of conc. sulfuric acid (56 mmol) and 105 ml of triethyl orthoformate (631 mmol) are initially introduced in a 1 l three-necked flask which has been rendered inert. The solution is heated under reflux for 30 min, then cooled to 0-4° C., and dimeric dihydroxyacetone is added every 12 hours in a total of 6 portions (6×4.23 g=25.4 g; 141 mmol). After a further 24 h at 0-4° C., 19 g of anhydrous sodium hydrogencarbonate (226 mmol) are added, and the mixture is stirred for a further 30 min. The reaction solution is subsequently filtered through Celite/silica gel at room temperature, and the filter cake is washed with 100 ml of ethyl acetate. After removal of the solvent in vacuo, the residue is taken up in 100 ml of ethyl acetate and re-distilled in vacuo. Addition of 400 ml of hexane causes the formation of a white solid, which is filtered and washed with 100 ml of hexane, giving 32.3 g of product (97%) as a white solid.

### Synthesis of 2,5-bis(benzyloxymethyl)-2,5-diethoxy-1,4-dioxane

20 g of 2,5-diethoxy-2,5-bis(hydroxymethyl)-1,4-dioxane (85 mmol) are dissolved in 106 ml of toluene and warmed to 45° C. 16.8 g of potassium hydroxide (255 mmol, 3 eq.) are then added, and 20.1 ml of benzyl bromide (16 mmol, 2 eq.) are slowly added dropwise. The mixture is subsequently heated under reflux for 4 hours. After cooling to 80° C., 100 ml of water are added, and the mixture is again heated under reflux for 16 h. After cooling to room temperature, the organic phase is separated off, and the solvent is removed in vacuo. The residue is subsequently recrystallised from 31 g of cold ethanol, giving 18.4 g (52%) of product as colourless crystals.

¹H-NMR (CDCl₃): δ=1.21 (t, 2×CH₃), 3.39 (d, CH₂—OBn, J=10.9 Hz), 3.53 (d, CH₂—OBn, J=10.9 Hz), 3.59 (d, 2×CH₂—CH₃), 3.64 (d, C—CH₂—O, J=11.7), 3.84 (d, C—CH₂—O, J=11.7), 4.53 (d, CH₂-Ph, J=12.1 Hz), 4.59 (d, CH₂-Ph, J=12.1 Hz), 7.27-7.45 (m, 5×Ar—H) ppm.

### Synthesis of 1-benzyloxy-3-hydroxypropan-2-one (Ia)

13 g of 2,5-bis(benzyloxymethyl)-2,5-diethoxy-1,4-dioxane (31 mmol) are dissolved in 62 ml of THF, and a mixture of 8.7 ml of 95% sulfuric acid with 31 ml of water is slowly added dropwise, during which the internal temperature is kept at 40° C. After a reaction time of 4 hours, 100 ml of MTBE are added to the two-phase reaction solution, and the organic phase is separated off. The solvent is removed in vacuo, and the residue is recrystallised from 50 ml of toluene/heptane 2/1, giving 7.2 g (64%) of product (Ia) as a solidifying oil.

¹H-NMR (DMSO-d₆): δ=3.39 (t, OH), 4.17 (d, CH₂—OH, J=5.9 Hz), 4.30 (s, CH₂—OCH₂Ph), 4.54 (s, Ph-CH₂O), 7.27-7.45 (m, 5×Ar—H) ppm.

### Example 1b

### Synthesis of Compound (Ic), n=7

### Synthesis of 2,5-diethoxy-2,5-bisheptyloxymethyl-1,4-dioxane

20 g of 2,5-diethoxy-2,5-bis(hydroxymethyl)-1,4-dioxane (85 mmol) are dissolved in 170 ml of toluene and warmed to 45° C. 16.8 g of potassium hydroxide (255 mmol, 3 eq.) are then added, and 40 ml of 1-bromoheptane (255 mmol, 3 eq.) are slowly added dropwise. The mixture is subsequently heated under reflux for 4 hours. After cooling to 80° C., 300 ml of water are added. After cooling to room temperature, the organic phase is separated off, and the solvent is removed in vacuo. The residue is subsequently purified by column chromatography (heptane/MTBE 1/1), giving 27.11 g (74%) of product as a pale-yellow oil.

¹H-NMR (500 MHz, CDCl₃): δ=0.88 (t, 2×CH₃), 1.18 (t, CH), 1.21-1.36 (m, 14×CH), 1.42 (m, 2×CH), 1.56 (m, 2×CH), 1.85 (q, 2×CH), 3.32-3.71 (m, 7×CH), 3.77 (d, C—CH₂—O, J=13.86), 3.85 (d, C—CH₂—O, J=13.86) ppm.

¹³C-NMR (75 MHz, CDCl₃): δ=14.03, 15.50, 22.62, 26.02, 26.08, 28.17, 28.45, 29.03, 29.11, 29.46, 29.53, 31.66, 31.83, 31.90, 32.88, 33.99, 55.64, 56.40, 56.60, 61.47, 63.92, 68.01, 68.74, 70.64, 71.52, 71.69, 71.91, 94.93, 98.00 ppm.

### Synthesis of 1-heptyloxy-3-hydroxypropan-2-one (Ic n=7)

19 g of 2,5-diethoxy-2,5-bisheptyloxymethyl-1,4-dioxane (43 mmol) are dissolved in 120 ml of THF, and a mixture of 13 ml of 95% sulfuric acid with 61 ml of water is slowly added dropwise, during which the internal temperature is kept at 40° C. After a reaction time of 48 hours, 122 ml of MTBE are added to the two-phase reaction solution, and the organic phase is separated off. The solvent is removed in vacuo, and the residue is purified by column chromatography (heptane/MTBE 1/1), giving 3.82 g (23%) of product (Ic n=7) as an oil.

¹H-NMR (DMSO-d₆): δ=0.89 (t, 2×CH₃), 1.28 (m, 8×CH), 1.60 (m, 2×CH), 3.45 (m, 3×CH) ppm.

### Example 2

### Tanning Experiment (Liquid Skin Model)

1 mmol of the self-tanner substance to be tested are dissolved in 94 ml of ethylene glycol and 6 ml of water (potassium hydrogenphthalate-buffered to pH=7) with 146 mg of DL-lysine (1 mmol) and stirred for 24 hrs. (=liquid skin model). The kinetics are documented via photos. After 24 h, the colour values (L*a*b* system) of the solution are measured by UV-VIS spectrometer (Varian Cary-50) in order to be able to assess the effect on the skin tones.

### Test Substances:

| | | Molecular | Sample |
|---|---|---|---|
| Abbreviation | Substance | weight | weight |
| DHA | dihydroxyacetone | 90 g/mol | 90 mg |
| MBDE | monobenzyl-DHA ether (Ia) | 180 g/mol | 180 mg |
| MHDE | monoheptyl-DHA ether (Ic) | 188 g/mol | 188 mg |
| MEHDE | mono-2-ethylhexyl-DHA ether (Ib) | 202.3 g/mol | 202 mg |
| MCHMDE | monocyclohexylmethyl-DHA ether | 188.3 g/mol | 188 mg |
| | (Id) | | |

### Kinetics:

Both MHDE and also MBDE exhibit a faster tanning reaction than DHA. An optical assessment gave approximately a doubling of the speed.

Lab values after 24 hours:

| | L* | a* | b* |
|---|---|---|---|
| DHA | 22.0 | 25.0 | 37.7 |
| MHDE (Ic) | 0.1 | 0.4 | 0.2 |
| MBDE (Ia) | 42.6 | 32.0 | 70.0 |
| DHA/MBDE 9/1 | 28.6 | 25.7 | 48.4 |
| DHA/MBDE 8/2 | 28.6 | 26.7 | 48.8 |
| DHA/MBDE 5/5 | 34.6 | 29.0 | 58.2 |
| MEHDE (Ib) | 86.5 | 4.7 | 24.3 |
| MCHMDE (Id) | 45.3 | 18.2 | 50.5 |

The Lab values show that MHDE (Ic) exhibits a darker coloration (lower L* value) and has more neutral colour values (a* and b* values close to 0) compared with DHA. MBDE (Ia) exhibits a strong shift towards a redder shade (higher a* than in the case of DHA), resulting in the formation of a more natural skin tone. In addition, the combination of MBDE (Ia) with DHA shows the positive effect with respect to the shade even at low proportions of MBDE. Furthermore, the intensity of the red shade is linear to the amount of MBDE employed, which facilitates accurate matching of the skin tone desired in each case to the specific wishes of the user.

MEHDE (Ib) exhibits a paler coloration (higher L* value) and has more neutral colour values (a* close to 0 and b* lower) compared with DHA.

MCHMDE (Id) exhibits a strong shift towards a more yellow shade (higher b* than in the case of DHA) and a slightly reduced red coloration.

### Solubilities:

In contrast to DHA, the DHA ethers exhibit good solubility in cosmetic oils (Finsolv TN as example):

### DHA: <<0.1%

### MBDE: 0.24%

### MEHDE: 2.25%

### MCHMDE: 0.97%.

### Example 3a

### O/W Tanning Cream

| | Source | | |
|---|---|---|---|
| Components/ | of | | |
| trade name | supply | INCI | [wt-%] |
| A | | | |
| Marlipal 1618/11 | (1) | CETEARETH-11 | 3.00 |
| Lanette O | (2) | CETEARYLALCOHOL | 7.00 |
| Luvitol EHO | (3) | CETEARYLOCTANOATE | 5.00 |
| Tegosoft TN | (4) | C12-15 ALKYLBENZOATE | 2.50 |
| Miglyol 812 N | (1) | CAPRYLIC/CAPRIC | 2.50 |
| | | TRIGLYCERIDE | |
| Propyl 4-hydroxy- | (5) | PROPYLPARABEN | 0.05 |
| benzoate | | | |
| MBDE | (5) | MONOBENZYL- | 5.00 |
| | | DIHYDROXYACETONETHER | |
| B | | | |
| 1,2-Propanediol | (5) | PROPYLENE GLYCOL | 4.00 |
| Methyl 4-hydroxy- | (5) | METHYLPARABEN | 0.15 |
| benzoate | | | |
| Water, | | AQUA (WATER) | 70.80 |
| demineralised | | | |

| | |
|---|---|
| Total | 100.00 |

### Preparation Process:

Firstly, phase A is heated to 75° C. and phase B to 80° C. Phase B is then slowly added to phase A with stirring and the mixture is stirred until a homogeneous mixture forms. After homogenisation, the formulation is stirred until cooled to room temperature. The pH is adjusted to the value pH=6.5 using sodium hydroxide solution or citric acid.

### Sources of Supply:

(1) Sasol Germany GmbH (2) Cognis GmbH (3) BASF AG (4) Degussa-Goldschmidt AG (5) Merck KGaA/Rona®

### Example 3b

### O/W Tanning Cream

| | Source | | |
|---|---|---|---|
| Components/ | of | | |
| trade name | supply | INCI | [wt-%] |
| A | | | |
| Marlipal 1618/11 | (1) | CETEARETH-11 | 3.00 |
| Lanette O | (2) | CETEARYLALCOHOL | 7.00 |
| Luvitol EHO | (3) | CETEARYLOCTANOATE | 5.00 |
| Tegosoft TN | (4) | C12-15 ALKYLBENZOATE | 2.50 |
| Miglyol 812 N | (1) | CAPRYLIC/CAPRIC | 2.50 |
| | | TRIGLYCERIDE | |
| Propyl 4-hydroxy- | (5) | PROPYLPARABEN | 0.05 |
| benzoate | | | |
| B | | | |
| 1,2-Propanediol | (5) | PROPYLENE GLYCOL | 4.00 |
| Methyl 4-hydroxy- | (5) | METHYLPARABEN | 0.15 |
| benzoate | | | |
| Water, | | AQUA (WATER) | 60.80 |
| demineralised | | | |
| C | | | |
| MHDE | (5) | MONOHEPTYL- | 5.00 |
| | | DIHYDROXYACETONETHER | |
| Ethanol 96% | (5) | ALCOHOL | 10.00 |

| | |
|---|---|
| Total | 100.00 |

### Preparation Process:

Firstly, phase A is heated to 75° C. and phase B to 80° C. Phase B is then slowly added to phase A with stirring and the mixture is stirred until a homogeneous mixture forms. After homogenisation and cooling of the emulsion, phase C is added at 40° C. The formulation is subsequently stirred until cooled to room temperature. The pH is adjusted to the value pH=5.5 using sodium hydroxide solution or citric acid.

### Sources of Supply:

(1) Sasol Germany GmbH (2) Cognis GmbH (3) BASF AG (4) Degussa-Goldschmidt AG (5) Merck KGaA/Rona®

### Example 4a

### O/W Tanning Cream

| | Source | | |
|---|---|---|---|
| Components/ | of | | |
| trade name | supply | INCI | [wt-%] |
| A | | | |
| Tego Care 150 | (1) | GLYCERYL STEARATE, | 8.00 |
| | | STEARETH-25-CETETH-20, | |
| | | STEARYL ALCOHOL | |
| Miglyol 812 N | (2) | CAPRYLIC/CAPRIC | 3.00 |
| | | TRIGLYCERIDE | |
| Isopropyl myristate | (3) | ISOPROPYL MYRISTATE | 2.00 |
| Paraffin liquid | (4) | PARAFFINUM LIQUIDUM | 12.00 |
| | | (MINERAL OIL) | |
| Paraffin | (4) | PARAFFIN | 2.00 |
| Propyl 4-hydroxy- | (4) | PROPYLPARABEN | 0.15 |
| benzoate | | | |
| MBDE | (5) | MONOBENZYL- | 5.00 |
| | | DIHYDROXYACETONETHER | |
| B | | | |
| 1,2-Propanediol | (4) | PROPYLENE GLYCOL | 4.00 |
| Sorbitol F liquid | (4) | SORBITOL | 2.00 |
| Methyl 4-hydroxy- | (4) | METHYLPARABEN | 0.05 |
| benzoate | | | |
| Water, | | AQUA (WATER) | 61.30 |
| demineralised | | | |
| C | | | |
| Fragrance (q.s.) | | PARFUM | 0.50 |

| | |
|---|---|
| Total | 100.00 |

### Preparation Process:

Firstly, phases A and B are warmed separately to 75° C. Phase A is then slowly added to phase B with careful stirring. The mixture is homogenised at 65° C. for C is added with stirring, and the mixture is cooled further. The pH is adjusted to the value pH=5.5 using sodium hydroxide solution or citric acid.

### Sources of Supply:

(1) Degussa-Goldschmidt AG (2) Sasol Germany GmbH (3) Cognis GmbH (4) Merck KGaA/Rona®

### Example 4b

### O/W Tanning Cream

| | Source | | |
|---|---|---|---|
| Components/ | of | | |
| trade name | supply | INCI | [wt-%] |
| A | | | |
| Tego Care 150 | (1) | GLYCERYL STEARATE, | 8.00 |
| | | STEARETH-25-CETETH-20, | |
| | | STEARYL ALCOHOL | |
| Miglyol 812 N | (2) | CAPRYLIC/CAPRIC | 3.00 |
| | | TRIGLYCERIDE | |
| Isopropyl myristate | (3) | ISOPROPYL MYRISTATE | 2.00 |
| Paraffin liquid | (4) | PARAFFINUM LIQUIDUM | 12.00 |
| | | (MINERAL OIL) | |
| Paraffin | (4) | PARAFFIN | 2.00 |
| Propyl 4-hydroxy- | (4) | PROPYLPARABEN | 0.15 |
| benzoate | | | |
| MBDE | (5) | MONOBENZYL- | 2.00 |
| | | DIHYDROXYACETONETHER | |
| B | | | |
| 1,2-Propanediol | (4) | PROPYLENE GLYCOL | 4.00 |
| Sorbitol F liquid | (4) | SORBITOL | 2.00 |
| Methyl 4-hydroxy- | (4) | METHYLPARABEN | 0.05 |
| benzoate | | | |
| Water, | | AQUA (WATER) | 47.50 |
| demineralised | | | |
| C | | | |
| Water, | | AQUA (WATER) | 11.80 |
| demineralised | | | |
| Dihydroxyacetone | (4) | DIHYDROXYACETONE | 5.00 |
| D | | | |
| Fragrance (q.s.) | | PARFUM | 0.50 |

| | |
|---|---|
| Total | 100.00 |

### Preparation Process:

Firstly, phases A and B are warmed separately to 75° C. Phase A is then slowly added to phase B with careful stirring. The mixture is homogenised at 65° C. for one minute. The mixture is subsequently cooled to 40° C. with stirring, and phase C is added. The mixture is then cooled to 35° C., and phase D is added with stirring, and the mixture is cooled further. The pH is adjusted to the value pH=6.0 using sodium hydroxide solution or citric acid.

### Sources of Supply:

(1) Degussa-Goldschmidt AG (2) Sasol Germany GmbH (3) Cognis GmbH (4) Merck KGaA/Rona®

### Example 5a

### O/W Tanning Cream

| | Source | | |
|---|---|---|---|
| Components/ | of | | |
| trade name | supply | INCI | [wt-%] |
| A | | | |
| Tego Care 150 | (1) | GLYCERYL STEARATE, | 8.00 |
| | | STEARETH-25, CETETH-20, | |
| | | STEARYL ALCOHOL | |
| Lanette O | (2) | CETEARYL ALCOHOL | 1.50 |
| Luvitol EHO | (3) | CETEARYL OCTANOATE | 5.00 |
| Miglyol 812 N | (4) | CAPRYLIC/CAPRIC | 5.00 |
| | | TRIGLYCERIDE | |
| Paraffin liquid | (5) | PARAFFINUM LIQUIDUM | 3.00 |
| | | (MINERAL OIL) | |
| AbilWax 2434 | (1) | STEAROXY DIMETHICONE | 1.60 |
| Dow Corning 200 | (6) | DIMETHICONE | 0.50 |
| Fluid (350 cs) | | | |
| Propyl 4-hydroxy- | (5) | PROPYLLPARABEN | 0.05 |
| benzoate | | | |
| B | | | |
| 1,2-Propanediol | (5) | PROPYLENE GLYCOL | 3.00 |
| Methyl 4-hydroxy- | (5) | METHYLPARABEN | 0.15 |
| benzoate | | | |
| Water, | | AQUA (WATER) | 52.20 |
| demineralised | | | |
| C | | | |
| MEHDE | (5) | MONOETHYLHEXYL- | 5.00 |
| | | DIHYDROXYACETONETHER | |
| Probiol L 05018 | (7) | AQUA, ALCOHOL DENAT, | 5.00 |
| (empty liposomes) | | LELCITHIN, GLYCERINE, | |
| | | DISODIUM PHOSPHATE | |
| Water, | | AQUA (WATER) | 10.00 |
| demineralised | | | |

| | |
|---|---|
| Total | 100.00 |

### Preparation Process:

Firstly, phases A and B are warmed to 80° C. Phase B is then slowly added to phase A with stirring, and the mixture is homogenised. The mixture is then cooled, and phase C is added at 40° C.

### Sources of Supply:

(1) Degussa-Goldschmidt AG (2) Cognis GmbH (3) BASF AG (4) Sasol Germany GmbH (5) Merck KGaA/Rona® (6) Dow Corning (7) Kuhs GmbH & Co. KG

### Example 5b

### O/W Tanning Cream

| | Source | | |
|---|---|---|---|
| Components/ | of | | |
| trade name | supply | INCI | [wt-%] |
| A | | | |
| Tego Care 150 | (1) | GLYCERYL STEARATE, | 8.00 |
| | | STEARETH-25, CETETH-20, | |
| | | STEARYL ALCOHOL | |
| Lanette O | (2) | CETEARYL ALCOHOL | 1.50 |
| Luvitol EHO | (3) | CETEARYL OCTANOATE | 5.00 |
| Miglyol 812 N | (4) | CAPRYLIC/CAPRIC | 5.00 |
| | | TRIGLYCERIDE | |
| Paraffin liquid | (5) | PARAFFINUM LIQUIDUM | 3.00 |
| | | (MINERAL OIL) | |
| AbilWax 2434 | (1) | STEAROXY DIMETHICONE | 1.60 |
| Dow Corning 200 | (6) | DIMETHICONE | 0.50 |
| Fluid (350 cs) | | | |
| Propyl 4-hydroxy- | (5) | PROPYLLPARABEN | 0.05 |
| benzoate | | | |
| MBDE | (5) | MONOBENZYL- | 1.00 |
| | | DIHYDROXYACETONETHER | |
| B | | | |
| 1,2-Propanediol | (5) | PROPYLENE GLYCOL | 3.00 |
| Methyl 4-hydroxy- | (5) | METHYLPARABEN | 0.15 |
| benzoate | | | |
| Water, | | AQUA (WATER) | 51.20 |
| demineralised | | | |
| C | | | |
| Dihydroxyacetone | (5) | DIHYDROXYACETONE | 5.00 |
| Probiol L 05018 | (7) | AQUA, ALCOHOL DENAT, | 5.00 |
| (empty liposomes) | | LELCITHIN, GLYCERINE, | |
| | | DISODIUM PHOSPHATE | |
| Water, | | AQUA (WATER) | 10.00 |
| demineralised | | | |

| | |
|---|---|
| Total | 100.00 |

### Preparation Process:

Firstly, phases A and B are warmed to 80° C. Phase B is then slowly added to phase A with stirring, and the mixture is homogenised. The mixture is then cooled, and phase C is added at 40° C.

### Sources of Supply:

(1) Degussa-Goldschmidt AG (2) Cognis GmbH (3) BASF AG (4) Sasol Germany GmbH (5) Merck KGaA/Rona® (6) Dow Corning (7) Kuhs GmbH & Co. KG

### Example 6a

### O/W Tanning Lotion

| | Source | | |
|---|---|---|---|
| Components/ | of | | |
| trade name | supply | INCI | [wt-%] |
| A | | | |
| Montanov 68 | (1) | CETEARYL ALCOHOL, | 4.00 |
| | | CETEARYL GLUCOSIDE | |
| Span 60 | (2) | SORBITAN STEARATE | 1.50 |
| Lanette O | (3) | CETEARYL ALCOHOL | 1.00 |
| Cosmacol ELI | (4) | C12-13 ALIKYL LACTATE | 2.00 |
| Arimol HD | (2) | ISOHEXADECANE | 1.50 |
| Paraffin highly | (5) | PARAFFINUM LIQUIDUM | 3.50 |
| liquid | | (MINERAL OIL) | |
| Dow Corning 9050 | (6) | CYCLOMETHICONE, | 2.00 |
| Silicone Elastomer | | DIMETHICONE | |
| Blend | | CROSSPOLYMER | |
| RonaCare ® | (5) | TOCOPHERYL ACETATE | 0.50 |
| Tocopherol Acetate | | | |
| Propyl 4-hydroxy- | (5) | PROPYLPARABEN | 0.05 |
| benzoate | | | |
| MHDE | (5) | MONOHEPTYL- | 2.50 |
| | | DIHYDROXYACETONETHER | |
| B | | | |
| RonaCare ® Ectoin | (5) | ECTOIN | 0.50 |
| Glycerine, | (5) | GLYCERINE | 2.00 |
| anhydrous | | | |
| Water, | | AQUA (WATER) | 60.90 |
| demineralised | | | |
| Methyl 4-hydroxy- | (5) | METHYLPARABEN | 0.15 |
| benzoate | | | |
| C | | | |
| Rhodicare S | (7) | XANTHAN GUM | 0.20 |
| D | | | |
| Probiol L 05018 | (8) | AQUA, ALCOHOL DENAT, | 5.00 |
| (empty liposomes) | | LECITHIN, GLYCERINE, | |
| | | DISODIUM PHOSPHATE | |
| Water, | | AQUA (WATER) | 10.00 |
| demineralised | | | |
| E | | | |
| Fragrance | (9) | PARFUM | 0.20 |
| cucumber | | | |

| | |
|---|---|
| Total | 100.00 |

### Preparation Process:

Firstly, phases A and B are mixed separately and warmed to 75° C. Phase C is then added to phase B and added to phase A with stirring. The mixture is homogenised. The mixture is then cooled with stirring, and phases D and E are added at 40° C.

### Sources of Supply:

(1) Seppic (2) Uniqema (3) Cognis GmbH (4) Condea Chinica D.A.C.S.p.A. (5) Merck KGaA/Rona® (6) Dow Corning (7) Rhodia GmbH (8) Kuhs GmbH & Co. KG (9) Drom

### Example 6b

### O/W Tanning Lotion

| | Source | | |
|---|---|---|---|
| Components/ | of | | |
| trade name | supply | INCI | [wt-%] |
| A | | | |
| Montanov 68 | (1) | CETEARYL ALCOHOL, | 4.00 |
| | | CETEARYL GLUCOSIDE | |
| Span 60 | (2) | SORBITAN STEARATE | 1.50 |
| Lanette O | (3) | CETEARYL ALCOHOL | 1.00 |
| Cosmacol ELI | (4) | C12-13 ALIKYL LACTATE | 2.00 |
| Arimol HD | (2) | ISOHEXADECANE | 1.50 |
| Paraffin highly | (5) | PARAFFINUM LIQUIDUM | 3.50 |
| liquid | | (MINERAL OIL) | |
| Dow Corning 9050 | (6) | CYCLOMETHICONE, | 2.00 |
| Silicone Elastomer | | DIMETHICONE | |
| Blend | | CROSSPOLYMER | |
| RonaCare ® | (5) | TOCOPHERYL ACETATE | 0.50 |
| Tocopherol Acetate | | | |
| Propyl 4-hydroxy- | (5) | PROPYLPARABEN | 0.05 |
| benzoate | | | |
| MEHDE | (5) | MONOETHYLHEXYL- | 5.00 |
| | | DIHYDROXYACETONETHER | |
| B | | | |
| RonaCare ® Ectoin | (5) | ECTOIN | 0.50 |
| Glycerine, | (5) | GLYCERINE | 2.00 |
| anhydrous | | | |
| Water, | | AQUA (WATER) | 60.90 |
| demineralised | | | |
| Methyl 4-hydroxy- | (5) | METHYLPARABEN | 0.15 |
| benzoate | | | |
| C | | | |
| Rhodicare S | (7) | XANTHAN GUM | 0.20 |
| D | | | |
| Probiol L 05018 | (8) | AQUA, ALCOHOL DENAT, | 5.00 |
| (empty liposomes) | | LECITHIN, GLYCERINE, | |
| | | DISODIUM PHOSPHATE | |
| Water, | | AQUA (WATER) | 10.00 |
| demineralised | | | |
| E | | | |
| Fragrance | (9) | PARFUM | 0.20 |
| Cucumber | | | |

| | |
|---|---|
| Total | 100.00 |

### Preparation Process:

Firstly, phases A and B are mixed separately and warmed to 75° C. Phase C is then added to phase B and added to phase A with stirring. The mixture is homogenised. The mixture is then cooled with stirring, and phases D and E are added at 40° C. The pH is adjusted to the value pH=6.0 using sodium hydroxide solution or citric acid.

### Sources of Supply:

(1) Seppic (2) Uniqema (3) Cognis GmbH (4) Condea Chinica D.A.C.S.p.A. (5) Merck KGaA/Rona® (6) Dow Corning (7) Rhodia GmbH (8) Kuhs GmbH & Co. KG (9) Drom

### Example 7a

### Mild Transparent W/O Tanning Lotion

| | Source | | |
|---|---|---|---|
| Components/ | of | | |
| trade name | supply | INCI | [wt-%] |
| A | | | |
| Dow Corning 3225 | (1) | CYCLOMETHICONE, | 23.60 |
| C | | DIMETHICONE COPOLYOL | |
| Propyl 4-hydroxy- | (2) | PROPYLPARABEN | 0.05 |
| benzoate | | | |
| MBDE | (2) | MONOBENZYL- | 2.00 |
| | | DIHYDROXYACETONETHER | |
| B | | | |
| Dihydroxyacetone | (2) | DIHYDROXYACETONE | 3.00 |
| Methyl 4-hydroxy- | (2) | METHYLPARABEN | 0.15 |
| benzoate | | | |
| 1,2-Propanediol | (2) | PROPYLENE GLYCOL | 35.90 |
| Water, | | AQUA (WATER) | 35.30 |
| demineralised | | | |

| | |
|---|---|
| Total | 100.00 |

### Preparation Process:

Firstly, phase B is dissolved and then added to phase A. The pH is adjusted to the value pH=6.0 using sodium hydroxide solution or citric acid.

### Sources of Supply:

(1) Dow Corning (2) Merck KGaA/Rona®

### Example 7b

### Mild Transparent W/O Tanning Lotion

| | Source | | |
|---|---|---|---|
| Components/ | of | | |
| trade name | supply | INCI | [wt-%] |
| A | | | |
| Dow Corning 3225 | (1) | CYCLOMETHICONE, | 23.60 |
| C | | DIMETHICONE COPOLYOL | |
| Propyl 4-hydroxy- | (2) | PROPYLPARABEN | 0.05 |
| benzoate | | | |
| B | | | |
| MHDE | (2) | MONOHEPTYL- | 5.00 |
| | | DIHYDROXYACETONETHER | |
| Methyl 4-hydroxy- | (2) | METHYLPARABEN | 0.15 |
| benzoate | | | |
| 1,2-Propanediol | (2) | PROPYLENE GLYCOL | 35.90 |
| Water, | | AQUA (WATER) | 35.30 |
| demineralised | | | |

| | |
|---|---|
| Total | 100.00 |

### Preparation Process:

Firstly, phase B is dissolved and then added to phase A.

### Sources of Supply:

(1) Dow Corning (2) Merck KGaA/Rona®

### Example 8a

### W/O Tanning Lotion

| | Source | | |
|---|---|---|---|
| Components/ | of | | |
| trade name | supply | INCI | [wt-%] |
| A | | | |
| Abil EM 97 | (1) | BIS-PEG/PPG-14/14 | 1.50 |
| | | DIMETHICONE, | |
| | | CYCLOPENTASILOXANE | |
| Abil EM 90 | (1) | CETYL PEG/PPG-10/1 | 1.30 |
| | | DIMETHICONE | |
| Ceraphyl 368 | (2) | ETHYLHEXYL PALMITATE | 2.00 |
| Tegosoft DEC | (1) | DIETHYLHEXYL | 5.00 |
| | | CARBONATE | |
| Dow Corning 345 | (3) | CYCLOMETHICONE | 13.00 |
| Dow Corning 9041 | (3) | DIMETHICONE | 3.00 |
| Silicone Elastomer | | CROSSPOLYMER, | |
| Blend | | DIMETHICONE | |
| Fragrance Babylon | (4) | PARFUM | 0.30 |
| MEHDE | (5) | MONOETHYLHEXYL- | 2.00 |
| | | DIHYDROXYACETONETHER | |
| B | | | |
| 1,2-Propanediol | (5) | PROPYLENE GLYCOL | 20.00 |
| Glycerine, | (5) | GLYCERINE | 3.00 |
| anhydrous | | | |
| Magnesium sulfate | (5) | MAGNESIUM SULPHATE | 2.00 |
| heptahydrate | | | |
| Phenonip | (6) | PHENOXYETHANOL, | 1.00 |
| | | BUTYLPARABEN, | |
| | | ETHYLPARABEN, | |
| | | PROPYLPARABEN, | |
| | | METHYLPARABEN | |
| Ethanol 96% | (5) | ALCOHOL | 8.00 |
| Dihydroxyacetone | (5) | DIHYDROXYACETONE | 3.00 |
| Water, | | AQUA (WATER) | 34.90 |
| demineralised | | | |

| | |
|---|---|
| Total | 100.00 |

### Preparation Process:

Firstly, the magnesium sulfate heptahydrate is dissolved in the water of phase B. The remaining components of phase B are then added. Phase B is slowly added to phase A with stirring. Stirring is continued rapidly for 2 minutes and the mixture is homogenised. The pH is adjusted to the value pH=5.5 using sodium hydroxide solution or citric acid.

### Sources of Supply:

(1) Degussa-Goldschmidt AG (2) ISP Global Technologies (3) Dow Corning (4) Drom (5) Merck KGaA/Rona® (6) Nipa Laboratorien GmbH

### Example 8b

### W/O Tanning Lotion

| | Source | | |
|---|---|---|---|
| Components/ | of | | |
| trade name | supply | INCI | [wt-%] |
| A | | | |
| Abil EM 97 | (1) | BIS-PEG/PPG-14/14 | 1.50 |
| | | DIMETHICONE, | |
| | | CYCLOPENTASILOXANE | |
| Abil EM 90 | (1) | CETYL PEG/PPG-10/1 | 1.30 |
| | | DIMETHICONE | |
| Ceraphyl 368 | (2) | ETHYLHEXYL PALMITATE | 2.00 |
| Tegosoft DEC | (1) | DIETHYLHEXYL | 5.00 |
| | | CARBONATE | |
| Dow Corning 345 | (3) | CYCLOMETHICONE | 13.00 |
| Dow Corning 9041 | (3) | DIMETHICONE | 3.00 |
| Silicone Elastomer | | CROSSPOLYMER, | |
| Blend | | DIMETHICONE | |
| Fragrance Babylon | (4) | PARFUM | 0.30 |
| MHDE | (5) | MONOHEPTYL- | 1.00 |
| | | DIHYDROXYACETONETHER | |
| B | | | |
| 1,2-Propanediol | (5) | PROPYLENE GLYCOL | 20.00 |
| Glycerine, | (5) | GLYCERINE | 3.00 |
| anhydrous | | | |
| Magnesium sulfate | (5) | MAGNESIUM SULPHATE | 2.00 |
| heptahydrate | | | |
| Phenonip | (6) | PHENOXYETHANOL, | 1.00 |
| | | BUTYLPARABEN, | |
| | | ETHYLPARABEN, | |
| | | PROPYLPARABEN, | |
| | | METHYLPARABEN | |
| Ethanol 96% | (5) | ALCOHOL | 8.00 |
| Water, | | AQUA (WATER) | 34.90 |
| demineralised | | | |
| Dihydroxyacetone | (5) | DIHYDROXYACETONE | 4.00 |

| | |
|---|---|
| Total | 100.00 |

### Preparation Process:

Firstly, the magnesium sulfate heptahydrate is dissolved in the water of phase B. The remaining components of phase B are then added. Phase B is slowly added to phase A with stirring. Stirring is continued rapidly for 2 minutes and the mixture is homogenised.

### Sources of Supply:

(1) Degussa-Goldschmidt AG (2) ISP Global Technologies (3) Dow Corning (4) Drom (5) Merck KGaA/Rona® (6) Nipa Laboratorien GmbH

### Example 10

### Aqueous/Alcoholic Tanning Lotion for Pump Sprays

| | Source | | |
|---|---|---|---|
| Components/ | of | | |
| trade name | supply | INCI | [wt-%] |
| MHDE | (1) | MONOHEPTYL- | 1.50 |
| | | DIHYDROXYACETONETHER | |
| Ethanol 96% | (1) | ALCOHOL | 40.00 |
| Tagat L 2 | (2) | PEG-20 GLYCERYL LAURATE | 7.00 |
| 1,2-Propanediol | (1) | PROPYLENE GLYCOL | 5.00 |
| Water, | | AQUA (WATER) | 42.50 |
| demineralised | | | |
| Dihydroxacetone | (1) | DIHYDROXYACETONE | 4.00 |

| | |
|---|---|
| Total | 100.00 |

### Preparation Process:

The MHDE is dissolved in the ethanol and the other components are added with stirring. The dihydroxyacetone is then added and homogenised.

### Sources of Supply:

(1) Merck KGaA/Rona® (2) Degussa Goldschmidt AG

### Example 11a

### W/Si Tanning Gel

| | Source | | |
|---|---|---|---|
| Components/ | of | | |
| trade name | supply | INCI | [wt-%] |
| A | | | |
| Dow Corning 5225 | (1) | CYCLOPEN TASILOXANE, | 20.00 |
| C | | PEG/PG-18/18 DIMETHICONE | |
| DM-Fluid-A-6cs | (2) | DIMETHICONE | 4.00 |
| Soybean Oil | (3) | GLYCINE SOJA (SOYBEAN | 2.00 |
| | | OIL) | |
| RonaCare ® | (4) | TOCOPHERYL ACETATE | 0.50 |
| Tocopherol Acetate | | | |
| MBDE | (4) | MONOBENZYL- | 2.00 |
| | | DIHYDROXYACETONETHER | |
| B | | | |
| RonaCare ® Ectoin | (4) | ECTOIN | 0.30 |
| 1,2-Propanediol | (4) | PROPYLENE GLYCOL | 30.00 |
| Dipropylene glycol | (5) | DIPROPYLENE GLYCOL | 10.00 |
| Sodium chloride | (4) | SODIUM CHLORIDE | 1.00 |
| Ethanol 96% | (4) | ALCOHOL | 5.00 |
| 1% caramel in | (6) | CARAMEL | 2.50 |
| water | | | |
| 1% FD&C Yellow | (5) | AQUA (WATER), CI 15985 | 0.25 |
| No. 6 in water | | (FD&C YELLOW NO. 6) | |
| Water, | | AQUA (WATER) | 19.25 |
| demineralised | | | |
| Dihydroxyacetone | (4) | DIHYDROXYACETONE | 3.00 |
| C | | | |
| Fragrance | (7) | PARFUM | 0.20 |
| Melopeach | | | |

| | |
|---|---|
| Total | 100.00 |

### Preparation Process:

Phase B is dissolved and added to phase A. Phases C and D are added successively with stirring. The mixture is homogenised. The pH is adjusted to the

### Sources of Supply:

(1) Dow Corning (2) S. Black GmbH (3) Gustav Heess GmbG (4) Merck KGaA/Rona® (5) BASF AG (6) D. D. Williamson (7) Drom

### Example 11b

### W/Si Tanning Gel

| | Source | | |
|---|---|---|---|
| Components/ | of | | |
| trade name | supply | INCI | [wt-%] |
| A | | | |
| Dow Corning 5225 | (1) | CYCLOPEN TASILOXANE, | 20.00 |
| C | | PEG/PG-18/18 DIMETHICONE | |
| DM-Fluid-A-6cs | (2) | DIMETHICONE | 4.00 |
| Soybean Oil | (3) | GLYCINE SOJA (SOYBEAN | 2.00 |
| | | OIL) | |
| RonaCare ® | (4) | TOCOPHERYL ACETATE | 0.50 |
| Tocopherol Acetate | | | |
| MHDE | (4) | MONOHEPTYL- | 5.00 |
| | | DIHYDROXYACETONETHER | |
| B | | | |
| RonaCare ® Ectoin | (4) | ECTOIN | 0.30 |
| 1,2-Propanediol | (4) | PROPYLENE GLYCOL | 30.00 |
| Dipropylene glycol | (5) | DIPROPYLENE GLYCOL | 10.00 |
| Sodium chloride | (4) | SODIUM CHLORIDE | 1.00 |
| Ethanol 96% | (4) | ALCOHOL | 5.00 |
| 1% caramel in | (6) | CARAMEL | 2.50 |
| water | | | |
| 1% FD&C Yellow | (5) | AQUA (WATER), CI 15985 | 0.25 |
| No. 6 in water | | (FD&C YELLOW NO. 6) | |
| Water, | | AQUA (WATER) | 19.25 |
| demineralised | | | |
| C | | | |
| Fragrance | (7) | PARFUM | 0.20 |
| Melopeach | | | |

| | |
|---|---|
| Total | 100.00 |

### Preparation Process:

Phase B is dissolved and added to phase A. Phase C is added successively with stirring. The mixture is homogenised.

### Sources of Supply:

(1) Dow Corning (2) S. Black GmbH (3) Gustav Heess GmbG (4) Merck KGaA/Rona® (5) BASF AG (6) D. D. Williamson (7) Drom

### Example 12a

### O/W Tanning Cream with UV A/B Protection

| | Source | | |
|---|---|---|---|
| Components/ | of | | |
| trade name | supply | INCI | [wt-%] |
| A | | | |
| Eusolex ® 2292 | (1) | ETHYLHEXYL | 3.00 |
| | | METHOXYCINNAMATE, | |
| | | BHT | |
| Eusolex ® 4360 | (1) | BENZOPHENONE-3 | 0.50 |
| Tego Care 150 | (2) | GLYCERYL STEARATE, | 8.00 |
| | | STEARETH-25, CETETH-20, | |
| | | STEARYL ALCOHOL | |
| Lanette O | (3) | CETEARYL ALCOHOL | 1.50 |
| Luvitol EHO | (4) | CETEARYL OCTANOATE | 5.00 |
| Miglyoll 812 N | (5) | CAPRYLIC/CAPRIC | 5.00 |
| | | TRIGLYCERIDE | |
| Paraffin liquid | (1) | PARAFFINUM LIQUIDUM | 3.00 |
| | | (MINERAL OIL) | |
| Abil-Wax 2434 | (2) | STEAROXY DIMETHICONE | 1.60 |
| Dow Corning 200 | (6) | DIMETHICONE | 0.50 |
| Fluid (350 cs) | | | |
| Propyl 4-hydroxy- | (1) | PROPYLPARABEN | 0.05 |
| benzoate | | | |
| MBDE | (1) | MONOBENZYL- | 1.00 |
| | | DIHYDROXYACETONETHER | |
| B | | | |
| 1,2-Propanediol | (1) | PROPYLENE GLYCOL | 3.00 |
| Methyl 4-hydroxy- | (1) | SODIUM METHYLPARABEN | 0.17 |
| benzoate sodium | | | |
| salt | | | |
| Dihydroxyacetone | (1) | DIHYDROXYACETONE | 5.00 |
| Water, | | AQUA (WATER) | 62.18 |
| demineralised | | | |

| | |
|---|---|
| Total | 100.00 |

### Preparation Process:

Firstly, phases A and B are mixed separately and warmed to 80° C. Phase B is then slowly added to phase A with stirring. The mixture is homogenised and cooled to room temperature. The pH is adjusted to the value pH=5.0 using sodium hydroxide solution or citric acid.

### Sources of Supply:

(1) Merck KGaA/Rona® (2) Degussa-Goldschmidt AG (3) Cognis GmbH (4) BASF AG (5) Sasol Germany GmbH (6) Dow Corning

### Example 12b

### O/W Tanning Cream with UV A/B Protection

| | Source | | |
|---|---|---|---|
| Components/ | of | | |
| trade name | supply | INCI | [wt-%] |
| A | | | |
| Eusolex ® 2292 | (1) | ETHYLHEXYL | 3.00 |
| | | METHOXYCINNAMATE, | |
| | | BHT | |
| Eusolex ® 4360 | (1) | BENZOPHENONE-3 | 0.50 |
| Tego Care 150 | (2) | GLYCERYL STEARATE, | 8.00 |
| | | STEARETH-25, CETETH-20, | |
| | | STEARYL ALCOHOL | |
| Lanette O | (3) | CETEARYL ALCOHOL | 1.50 |
| Luvitol EHO | (4) | CETEARYL OCTANOATE | 5.00 |
| Miglyol 812 N | (5) | CAPRYLIC/CAPRIC | 5.00 |
| | | TRIGLYCERIDE | |
| Paraffin liquid | (1) | PARAFFINUM LIQUIDUM | 3.00 |
| | | (MINERAL OIL) | |
| Abil-Wax 2434 | (2) | STEAROXY DIMETHICONE | 1.60 |
| Dow Corning 200 | (6) | DIMETHICONE | 0.50 |
| Fluid (350 cs) | | | |
| Propyl 4-hydroxy- | (1) | PROPYLPARABEN | 0.05 |
| benzoate | | | |
| MBDE | (1) | MONOBENZYL- | 3.00 |
| | | DIHYDROXYACETONETHER | |
| B | | | |
| 1,2-Propanediol | (1) | PROPYLENE GLYCOL | 3.00 |
| Methyl 4-hydroxy- | (1) | SODIUM METHYLPARABEN | 0.17 |
| benzoate sodium | | | |
| salt | | | |
| Water, | | AQUA (WATER) | 53.18 |
| demineralised | | | |
| C | | | |
| Dihydroxyacetone | (1) | DIHYDROXYACETONE | 2.00 |
| Water, | | AQUA (WATER) | 10.00 |
| demineralised | | | |

| | |
|---|---|
| Total | 100.00 |

### Preparation Process:

Firstly, phases A and B are mixed separately and warmed to 80° C. Phase B is then slowly added to phase A with stirring. The mixture is homogenised. The mixture is subsequently cooled with stirring, and phase C is added at 40° C.

### Sources of Supply:

(1) Merck KGaA/Rona® (2) Degussa-Goldschmidt AG (3) Cognis GmbH (4) BASF AG (5) Sasol Germany GmbH (6) Dow Corning

### Example 12c

### O/W Tanning Cream with UV A/B Protection

| | Source | | |
|---|---|---|---|
| Components/ | of | | |
| trade name | supply | INCI | [wt-%] |
| A | | | |
| Eusolex ® 2292 | (1) | ETHYLHEXYL | 3.00 |
| | | METHOXYCINNAMATE, | |
| | | BHT | |
| Eusolex ® 4360 | (1) | BENZOPHENONE-3 | 0.50 |
| Tego Care 150 | (2) | GLYCERYL STEARATE, | 8.00 |
| | | STEARETH-25, CETETH-20, | |
| | | STEARYL ALCOHOL | |
| Lanette O | (3) | CETEARYL ALCOHOL | 1.50 |
| Luvitol EHO | (4) | CETEARYL OCTANOATE | 5.00 |
| Miglyol 812 N | (5) | CAPRYLIC/CAPRIC | 5.00 |
| | | TRIGLYCERIDE | |
| Paraffin liquid | (1) | PARAFFINUM LIQUIDUM | 3.00 |
| | | (MINERAL OIL) | |
| Abil-Wax 2434 | (2) | STEAROXY DIMETHICONE | 1.60 |
| Dow Corning 200 | (6) | DIMETHICONE | 0.50 |
| Fluid (350 cs) | | | |
| Propyl 4-hydroxy- | (1) | PROPYLPARABEN | 0.05 |
| benzoate | | | |
| MEHDE | (1) | MONOETHYLHEXYL- | 7.00 |
| | | DIHYDROXYACETONETHER | |
| B | | | |
| 1,2-Propanediol | (1) | PROPYLENE GLYCOL | 3.00 |
| Methyl 4-hydroxy- | (1) | SODIUM METHYLPARABEN | 0.17 |
| benzoate sodium | | | |
| salt | | | |
| Water, | | AQUA (WATER) | 51.18 |
| demineralised | | | |
| C | | | |
| Water, | | AQUA (WATER) | 10.00 |
| demineralised | | | |

| | |
|---|---|
| Total | 100.00 |

### Preparation Process:

Firstly, phases A and B are mixed separately and warmed to 80° C. Phase B is then slowly added to phase A with stirring. The mixture is homogenised. The mixture is subsequently cooled with stirring, and phase C is added at 40° C.

### Sources of Supply:

(1) Merck KGaA/Rona® (2) Degussa-Goldschmidt AG (3) Cognis GmbH (4) BASF AG (5) Sasol Germany GmbH (6) Dow Corning

### Example 12d

### O/W Tanning Cream with UV A/B Protection

| | Source | | |
|---|---|---|---|
| Components/ | of | | |
| trade name | supply | INCI | [wt-%] |
| A | | | |
| Eusolex ® 2292 | (1) | ETHYLHEXYL | 3.00 |
| | | METHOXYCINNAMATE, | |
| | | BHT | |
| Eusolex ® 4360 | (1) | BENZOPHENONE-3 | 0.50 |
| Tego Care 150 | (2) | GLYCERYL STEARATE, | 8.00 |
| | | STEARETH-25, CETETH-20, | |
| | | STEARYL ALCOHOL | |
| Lanette O | (3) | CETEARYL ALCOHOL | 1.50 |
| Luvitol EHO | (4) | CETEARYL OCTANOATE | 5.00 |
| Miglyol 812 N | (5) | CAPRYLIC/CAPRIC | 5.00 |
| | | TRIGLYCERIDE | |
| Paraffin liquid | (1) | PARAFFINUM LIQUIDUM | 3.00 |
| | | (MINERAL OIL) | |
| Abil-Wax 2434 | (2) | STEAROXY DIMETHICONE | 1.60 |
| Dow Corning 200 | (6) | DIMETHICONE | 0.50 |
| Fluid (350 cs) | | | |
| Propyl 4-hydroxy- | (1) | PROPYLPARABEN | 0.05 |
| benzoate | | | |
| MBDE | (1) | MONOBENZYL- | 1.00 |
| | | DIHYDROXYACETONETHER | |
| B | | | |
| 1,2-Propanediol | (1) | PROPYLENE GLYCOL | 3.00 |
| Methyl 4-hydroxy- | (1) | SODIUM METHYLPARABEN | 0.17 |
| benzoate sodium | | | |
| salt | | | |
| Water, | | AQUA (WATER) | 52.18 |
| demineralised | | | |
| C | | | |
| Dihydroxyacetone | (1) | DIHYDROXYACETONE | 5.00 |
| Water, | | AQUA (WATER) | 10.00 |
| demineralised | | | |

| | |
|---|---|
| Total | 100.00 |

### Preparation Process:

Firstly, phases A and B are mixed separately and warmed to 80° C. Phase B is then slowly added to phase A with stirring. The mixture is homogenised. The mixture is subsequently cooled with stirring, and phase C is added at 40° C. The pH is adjusted to the value pH=5.0 using sodium hydroxide solution or citric acid.

### Sources of Supply:

(1) Merck KGaA/Rona® (2) Degussa-Goldschmidt AG (3) Cognis GmbH (4) BASF AG (5) Sasol Germany GmbH (6) Dow Corning

### Example 12e

### O/W Tanning Cream with UV A/B Protection

| | Source | | |
|---|---|---|---|
| Components/ | of | | |
| trade name | supply | INCI | [wt-%] |
| A | | | |
| Eusolex ® 2292 | (1) | ETHYLHEXYL | 3.00 |
| | | METHOXYCINNAMATE, | |
| | | BHT | |
| Eusolex ® 4360 | (1) | BENZOPHENONE-3 | 0.50 |
| Tego Care 150 | (2) | GLYCERYL STEARATE, | 8.00 |
| | | STEARETH-25, CETETH-20, | |
| | | STEARYL ALCOHOL | |
| Lanette O | (3) | CETEARYL ALCOHOL | 1.50 |
| Luvitol EHO | (4) | CETEARYL OCTANOATE | 5.00 |
| Miglyol 812 N | (5) | CAPRYLIC/CAPRIC | 5.00 |
| | | TRIGLYCERIDE | |
| Paraffin liquid | (1) | PARAFFINUM LIQUIDUM | 3.00 |
| | | (MINERAL OIL) | |
| Abil-Wax 2434 | (2) | STEAROXY DIMETHICONE | 1.60 |
| Dow Corning 200 | (6) | DIMETHICONE | 0.50 |
| Fluid (350 cs) | | | |
| Propyl 4-hydroxy- | (1) | PROPYLPARABEN | 0.05 |
| benzoate | | | |
| MBDE | (1) | MONOBENZYL- | 1.00 |
| | | DIHYDROXYACETONETHER | |
| B | | | |
| 1,2-Propanediol | (1) | PROPYLENE GLYCOL | 3.00 |
| Methyl 4-hydroxy- | (1) | SODIUM METHYLPARABEN | 0.17 |
| benzoate sodium | | | |
| salt | | | |
| Water, | | AQUA (WATER) | 63.18 |
| demineralised | | | |
| Dihydroxyacetone | (1) | DIHYDROXYACETONE | 4.00 |

| | |
|---|---|
| Total | 100.00 |

### Preparation Process:

Firstly, phases A and B are mixed separately and warmed to 80° C. Phase B is then slowly added to phase A with stirring. The mixture is homogenised.

### Sources of Supply:

(1) Merck KGaA/Rona® (2) Degussa-Goldschmidt AG (3) Cognis GmbH (4) BASF AG (5) Sasol Germany GmbH (6) Dow Corning

### Example 13

### O/W Shimmering Tanning Lotion

| | Source | | |
|---|---|---|---|
| Components/ | of | | |
| trade name | supply | INCI | [wt-%] |
| A | | | |
| Montanov 68 | (1) | CETEARYL ALCOHOL, | 4.00 |
| | | CETEARYL GLUCOSIDE | |
| Span 60 | (2) | SORBITAN STEARATE | 1.50 |
| Lanette O | (3) | CETEARYL ALCOHOL | 1.00 |
| Cosmacol ELI | (4) | C12-13 ALKYL LACTATE | 3.00 |
| Cosmacol EMI | (4) | DI-C12-13 ALKYL MALATE | 1.50 |
| Dow Corning 9040 | (5) | CYCLOMETHICONE, | 1.00 |
| Silicone Elastomer | | DIMETHICONE | |
| Blend | | CROSSPOLYMER | |
| Arlamol HD | (2) | ISOHEXADECANE | 3.00 |
| RonaCare ® | (6) | TOCOPHERYL ACETATE | 0.50 |
| Tocopherol Acetate | | | |
| Propyl 4-hydroxy- | (6) | PROPYLPARABEN | 0.05 |
| benzoate | | | |
| MBDE | (6) | MONOBENZYL- | 2.00 |
| | | DIHYDROXYACETONETHER | |
| | | | 0.50 |
| B | | | |
| RonaCare ® Ectoin | (6) | ECTOIN | 0.50 |
| Colorona ® Red | (6) | MICA, CI 77891 (TITANIUM | 2.00 |
| Gold | | DIOXIDE), CI 77491 (ION | |
| | | OXIDES) | |
| Glycerine, | (6) | GLYCERIN | 2.00 |
| anhydrous | | | |
| Caramel 250 | (7) | CARAMEL | 1.00 |
| FD&C Yellow No6 | (8) | CI 15985 | 0.01 |
| W082 | | | |
| Water, | | AQUA (WATER) | 73.09 |
| demineralised | | | |
| Methyl 4-hydroxy- | (6) | METHYLPARABEN | 0.15 |
| benzoate | | | |
| DHA Plus | (6) | DIHYDROXYACETONE, | 3.00 |
| | | SODIUM METABISULFITE, | |
| | | MAGNESIUM STEARATE | |
| C | | | |
| Sepigel 305 | (1) | LAURETH-7, POLYACRYL- | 0.50 |
| | | AMIDE, C13-14 | |
| | | ISOPARAFFIN | |
| D | | | |
| Fragrance Babylon | (9) | PARFUM | 0.20 |

| | |
|---|---|
| Total | 100.00 |

### Preparation Process:

Firstly, phases A and B are warmed separately to 75° C. Phase A is then slowly added to phase B with stirring. At 60° C., phase C is added to A/B, and the mixture is homogenised. The mixture is subsequently cooled to 40° C., and phase D is added successively.

### Sources of Supply:

(1) Seppic (2) Uniqema (3) Cognis GmbH (4) Condea Chimica D.A.C.S.p.A. (5) Dow Corning (6) Merck KGaA/Rona® (7) D. D. Williamson (8) Les Colorants Wackherr SA (9) Drom

### Example 14

### Day-Care Cream

| | Source | | |
|---|---|---|---|
| Components/ | of | | |
| trade name | supply | INCI | [wt-%] |
| A | | | |
| Tego Care 150 | (1) | GLYCERYL STEARATE, | 8.00 |
| | | STEARETH-25, CETETH-20, | |
| | | STEARYL ALCOHOL | |
| Lanette O | (2) | CETEARYL ALCOHOL | 1.50 |
| Tegosoft liquid | (1) | CETEARYL | 6.50 |
| | | ETHYLHEXANOATE | |
| Miglyol 812 N | (3) | CAPRYLIC/CAPRIC | 6.50 |
| | | TRIGLYCERIDE | |
| Abil-Wax 2434 | (1) | STEAROXY DIMETHICONE | 1.20 |
| Dow Corning 200 | (4) | DIMETHICONE | 0.50 |
| (100 cs) | | | |
| RonaCare ® | (5) | TOCOPHERYL ACETATE | 0.50 |
| Tocopherol Acetate | | | |
| Propyl 4-hydroxy- | (5) | PROPYLPARABEN | 0.05 |
| benzoate | | | |
| MBDE | (5) | MONOBENZYL- | 2.00 |
| | | DIHYDROXYACETONETHER | |
| B | | | |
| 1,2-Propanediol | (5) | | 3.00 |
| Methyl 4-hydroxy- | (5) | PROPYLENE GLYCOL | 0.15 |
| benzoate | | | |
| Water, | | METHYLPARABEN | 57.00 |
| demineralised | | | |
| | | AQUA (WATER) | |
| C | | | |
| DHA-Rapid | (5) | DIHYDROXYACETONE, | 3.00 |
| | | TROXERUTIN | |
| Water, | | AQUA (WATER) | 10.00 |
| demineralised | | | |
| D | | | |
| Fragrance | (6) | PARFUM | 0.10 |
| “sunshine for | | | |
| cream D” | | | |

| | |
|---|---|
| Total | 100.00 |

### Preparation Process:

Firstly, phases A and B are warmed separately to 80° C. Phase B is then slowly added to phase A with stirring. The mixture is homogenised. The mixture is subsequently cooled with stirring, and phase C is added at 40° C. Phase D is added.

### Sources of Supply:

(1) Degussa-Goldschmidt AG (2) Cognis GmbH (3) Sasol Germany GmbH (4) Dow Corning (5) Merck KGaA/Rona® (6) Bell Flavors & Fragrances

1. A method for self-tanning which comprises applying a composition containing a compound of the formula (I) 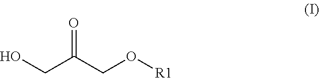
as self-tanning substance,
where R1 stands for a branched or unbranched alkyl radical having 1 to 20 C atoms, a branched or unbranched alkenyl radical having 2 to 20 C atoms or a branched or unbranched alkynyl radical having 2 to 20 C atoms,
where one or more CH₂ groups which are not adjacent and are not directly adjacent to the O atom of the formula (I) may be replaced by C═O or —O—,
where the radicals may be substituted by one or more OH groups, by one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms.

2. Method according to claim 1, characterised in that R1 stands for a branched or unbranched alkyl radical having 1 to 20 C atoms, where one or more CH₂ groups which are not adjacent and are not directly adjacent to the O atom of the formula (I) may be replaced by C═O or —O—, where the radicals may be substituted by one or more OH groups, by one or more cyclic alkyl radicals having 3 to 8 C atoms and/or by one or more aromatic ring systems having 5 to 6 C atoms, where the one or more aromatic ring systems may be substituted by one or more groups selected from —OH and —OR, and where R is as defined in claim 1.

3. Method according to claim 1, characterised in that R1 stands for a branched or unbranched alkyl radical having 1 to 20 C atoms,
where one or more CH₂ groups which are not adjacent and are not directly adjacent to the O atom of the formula (I) may be replaced by C═O or —O—,
where the radicals may be substituted by one or more OH groups, by one or more cyclic alkyl radicals having 5 to 6 C atoms and/or by one or more aromatic ring systems having 5 to 6 C atoms, where the one or more aromatic ring systems may be substituted by one or more groups —OR,
and where R is as defined in claim 1.

4. Method according to claim 1, characterised in that the compound of the formula (I) is selected from the compounds of the formula (Ia) to (Ik)

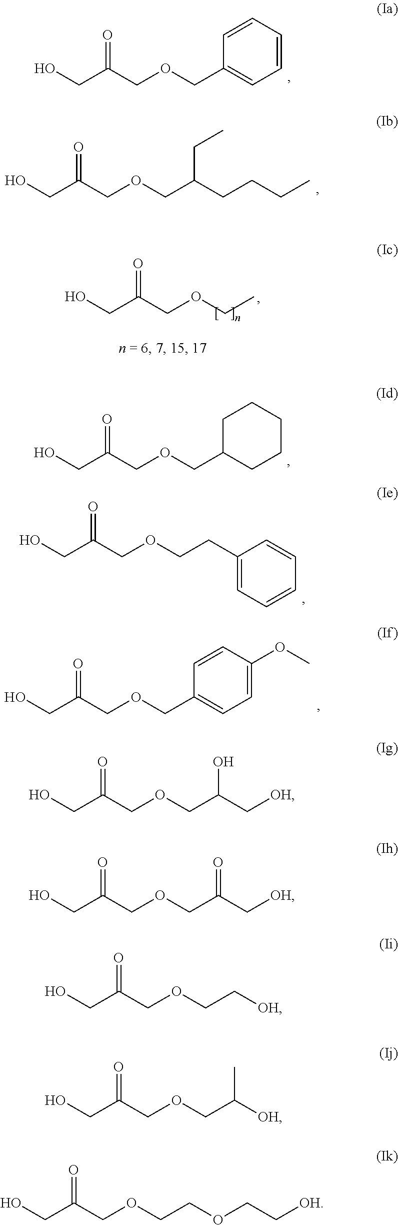

5. Method according to claim 1, characterised in that the applying takes place together with DHA and/or erythrulose.

6. Composition comprising at least one compound of the formula (I) 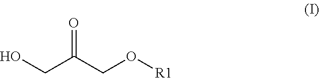
where R1 stands for a branched or unbranched alkyl radical having 1 to 20 C atoms, a branched or unbranched alkenyl radical having 2 to 20 C atoms or a branched or unbranched alkynyl radical having 2 to 20 C atoms,
where one or more CH₂ groups which are not adjacent and are not directly adjacent to the O atom of the formula (I) may be replaced by C═O or —O—,
where these radicals may be substituted by one or more OH groups, one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms.

7. Composition according to claim 6, characterised in that the preparation comprises at least one compound of the formula (I) in an amount of 0.01 to 20% by weight, based on the total amount of the preparation.

8. Composition according to claim 6, characterised in that the preparation comprises at least one further self-tanning substance.

9. Composition according to claim 8, characterised in that the self-tanning substance is selected from DHA and erythrulose.

10. Composition according to claim 6, characterised in that the preparation additionally comprises at least one UV filter.

11. Process for the preparation of a composition according to claim 6, characterised in that the at least one compound of the formula (I) is mixed with at least one vehicle which is suitable for topical preparations and optionally with assistants and/or fillers.

12. Compounds of the formula (I) 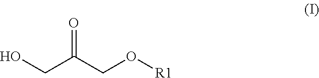
where R1 stands for a branched or unbranched alkyl radical having 1 to 20 C atoms, a branched or unbranched alkenyl radical having 2 to 20 C atoms or a branched or unbranched alkynyl radical having 2 to 20 C atoms,
where one or more CH₂ groups which are not adjacent and are not directly adjacent to the O atom of the formula (I) may be replaced by C═O or —O—,
where these radicals may be substituted by one or more OH groups, one or more cyclic alkyl radicals having 3 to 8 C atoms and/or by one or more aromatic ring systems having 5 to 6 C atoms, where the one or more aromatic ring systems may be substituted by one or more groups selected from —OH, —OR, —NR₂, —CN, —COOR and —COOR′;
where R stands on each occurrence, independently of one another, for a branched or unbranched alkyl radical having 1 to 20 C atoms;
and where R′ stands for an aromatic ring system having 5 or 6 C atoms;
and where the following compounds are excluded:

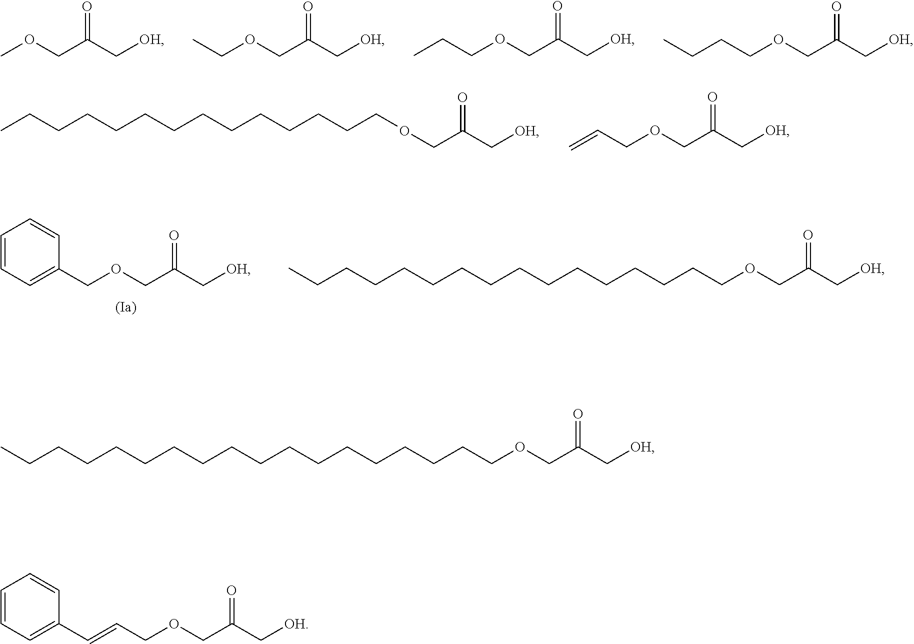

13. Compounds of the formula (I) according to claim 12, characterised in that the compounds are selected from the compounds of the formulae (Ib) to (Ik)

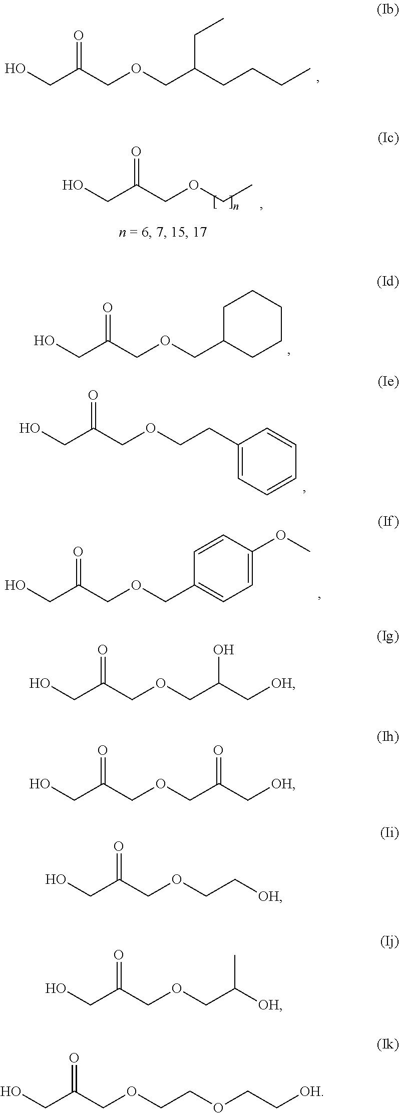

14. Process for the preparation of a compound of the formula (I) according to claim 12, characterised in that 2,5-diethoxy-2,5-bis(hydroxymethyl)-1,4-dioxane is reacted with a compound of the formula R1-X and subsequently subjected to acidic hydrolysis, where X stands for Cl, Br, I, OSO₂CH₃ (O-mesyl) or OSO₂C₆H₄CH₃ (Otosyl).
